(19) United States
(12) Patent Application Publication
(10) Pub. No.: US 2024/0138419 A1
(43) Pub. Date: May 2, 2024

(54) FORMULATIONS, METHODS AND USES THEREOF

(71) Applicant: N&E INNOVATIONS PTE. LTD, Singapore (SG)

(72) Inventors: Jie Lin Jaslyn LEE, Singapore (SG); Ming Quan Didi GAN, Singapore (SG)

(21) Appl. No.: 18/279,676

(22) PCT Filed: Mar. 1, 2022
(86) PCT No.: PCT/SG2022/050099

§ 371 Date: Sep. 1, 2023

(30) Foreign Application Priority Data
Mar. 1, 2021 (SG) 10202102065Y

(51) Int. Cl. A01N 65/08; A01N 25/24; A01N 59/16; A01P 1/00; C11D 3/12; C11D 3/382; C11D 3/48; C11D 9/00

(52) U.S. Cl. CPC A01N 65/08; A01N 25/24; A01N 59/16; A01P 1/00; C11D 3/1206; C11D 3/382; C11D 3/48; C11D 9/00; C11D 2111/12

(57) ABSTRACT

The present invention relates to anti-bacterial and anti-viral formulations comprising cashew testa extract, an iron particle and/or iron oxide particle, and a carbohydrate; and wherein the cashew testa extract is at least partially incorporated in the iron particle and/or iron oxide particle. A weight ratio of the cashew testa extract to the iron particle and/or iron oxide particle is 100:1 to 1:200; and a weight ratio of the carbohydrate to the cashew testa extract and iron particle and/or iron oxide particle is 8:1 to 1:300.

### TECHNICAL FIELD

The present invention relates, in general terms, to formulations for use in anti-microbial applications and methods of formulating thereof. In particular, the present invention relates to anti-bacterial and anti-viral formulations made from natural products and their uses thereof.

### BACKGROUND

Microbial infections and the development of antimicrobial resistance have recently been receiving attention as one of the more important critical issues faced by public health authorities. Microbiological contamination refers to the non-intended or accidental introduction of microbes such as bacteria, yeast, mould, fungi, virus, prions, protozoa or their toxins and by-products. There is also rising concerns regarding the increase in Hospital-Acquired Infections (HAIs) cases.

Diseases caused by zoonotic highly pathogenic viruses are also of increasing concern to health authorities. Prominent recent examples are the outbreaks of diseases caused by the SARS and MERS coronaviruses (SARS-CoV, MERS-CoV), the avian influenza virus (AIV) and Ebola virus (EBOV). With increasing globalization, the threat of the diseases caused by such highly pathogenic viruses has been amplified. In some cases, there is the added threat that the viruses could be used for bioterrorism.

Highly pathogenic viruses (HP viruses) includes all viruses where there is currently no vaccine and where the viruses are capable of causing fatal systemic disease in humans if untreated and/or which might have the potential to be used as bioterror weapons.

As a specific example, and more recently, coronavirus disease (COVID-19) is an infectious disease caused by a new virus. The disease causes respiratory illness (like the flu) with symptoms such as a cough, fever, and in more severe cases, difficulty breathing.

Apart from the fact that all these viruses are capable of causing fatal illness in humans and there is not yet available an effective treatment or vaccine, the other important commonality is that they are all zoonotic, i.e. originally transmitted from animals to humans. By way of example, the animal source for AIV is birds, for EBOV it is fruit bats, and for COVID-19 is bats.

Another common feature is that they have relatively high mortality rates in humans. In some cases, such as avian influenza, they have the potential to become pandemic, causing large numbers of deaths and major challenges for frontline healthcare workers who are at risk of contracting the virus. A further concern is that some of the viruses responsible for these diseases may have the capacity to be developed as bioterror weapons.

Such diseases (for example coronavirus disease) spreads primarily through contact with an infected person when they cough or sneeze. It also spreads when a person touches a surface or object that has the virus on it, then touches their eyes, nose, or mouth. Accordingly, there is a need for surface or objects, especially in public areas, to be sanitised or cleaned.

The growing concern regarding cleanliness in various industries has led to an increased demand for antimicrobial products. These can be used to protect surfaces against microorganisms and has applications in medical devices and packaging. The demand for antimicrobial additives is also rapidly expanding due to growing population and urbanization.

Towards this end, the global antimicrobial coatings market size was valued at USD 7.1 billion in 2019 and is expected to grow at a compound annual growth rate (CAGR) of 12.8% from 2020 to 2027.

Current anti-microbial products in the market rely on titanium, zinc and silver compounds to impart the anti-microbial effect. While effective, such elements are expensive and are also toxic and prone to heavy metal contamination. The anti-microbial efficacy is also low due to its dependence on photo irradiation.

There are further concerns regarding whether such products are toxic to humans and animals. Further, when harsh chemicals are used during the synthesis, there is always a possibly that the toxic precursors such as heavy metals are retained in the final products such that extensive washing is required. This increases production cost.

In Singapore, one of the biggest waste streams is food waste. In 2019, up to 744 million kg of food waste was generated. This puts a strain on the waste treatment facilities and limited landfill facilities in land scarce Singapore. In addition, food wastage also increases the overall carbon footprint, as the resources used to grow and import the food is wasted. There is a need to combat this emerging food waste problem and ensure sustainability of our environment and ecosystem.

It would be desirable to overcome or ameliorate at least one of the above-described problems.

### SUMMARY

The present invention is predicated on the understanding that certain natural products are anti-microbial and can act synergistically (or at least additively) when combined in a formulation. In particular, the inventors have found that an extract of cashew testa is advantageous in that it has an anti-microbial efficacy. For example, the extract was found to possess anti-bacterial properties against gram negative bacteria Escherichia coli and gram positive bacteria Staphylococcus aureus. The natural product extract can be applied in a green solvent and can have a penetrative effect when tested on surfaces and textile. The extract was also found to have anti-viral properties. Further, when combined with other components such as iron particles and/or iron oxide particles, a synergistic (or at least additive) anti-microbial effect was observed. For example, the cashew testa extract can be used as a precursor for synthesising iron particles and/or iron oxide particles. Further, when combined with a carbohydrate, a further synergistic (or at least additive) anti-microbial effect can be obtained with an improved retention time of radicals. As these formulations are synthesised/made from biological or natural materials, they can be safe for human and animals. Such green biosynthesis is cost effective with less waste production, non-toxic, and environmentally friendly. The present invention has applications as an antimicrobial coating, disinfectant, hand sanitizer, and/or soap.

The present invention provides a formulation, comprising:
a) cashew testa extract;
b) an iron particle and/or iron oxide particle; and
c) a carbohydrate;
wherein a weight ratio of the cashew testa extract to the iron particle and/or iron oxide particle is about 100:1 to about 1:200; and
wherein a weight ratio of the carbohydrate (c) to the cashew testa extract and iron particle and/or iron oxide particle (a and b) is about 8:1 to about 1:300; and
wherein the cashew testa extract is at least partially incorporated in the iron particle and/or iron oxide particle.

Advantageously, when formulated in this ratio, the carbohydrate improves the retention time of the radicals produced by the cashew testa extract and iron particle and/or iron oxide particle on the applied surface. Further, the carbohydrate improves the adherence of the cashew testa extract and iron particle and/or iron oxide particle on applied surface. Accordingly, a synergistic effect (or at least additive) in anti-microbial activity is observed.

In some embodiments, the weight ratio of the carbohydrate (c) to the cashew testa extract and iron particle and/or iron oxide particle (a and b) is about 8:1 to about 1:1.

In some embodiments, the weight ratio of the carbohydrate is about 1 wt % to about 15 wt % relative to the formulation.

In some embodiments, the carbohydrate is selected from chitosan.

In some embodiments, the weight ratio of the cashew testa extract and iron particle and/or iron oxide particle (a and b) is about 0.5 wt % to about 8 wt % relative to the formulation.

In some embodiments, the formulation has a pH of about 4 to about 5.

In some embodiments, the formulation further comprises an excipient selected from colorant, humectant, fragrance, stabilizer, permeabilizer, attachment promoter, film forming agent, transfection agent, surfactant, solvent, anti-oxidant or a combination thereof.

In some embodiments, the formulation further comprises a colorant selected from β-carotene, asthaxanthin or a combination thereof.

Advantageously, the colorant provides a visual cue to a user that a surface is coated with an anti-microbial coating. This provides confidence to the user and also provides an indication for reapplication of the coating if required.

In some embodiments, a weight ratio of the colorant is about 0.01 wt % to about 10 wt % relative to the formulation.

In some embodiments, the formulation further comprises a humectant selected from glycerine, urea, pyrrolidine carboxylic acid, aloe vera or a combination thereof.

In some embodiments, a weight ratio of the humectant is about 2 wt % to about 60 wt % relative to the formulation.

In some embodiments, the formulation further comprises a fragrance, wherein the fragrance comprises an essential oil.

In some embodiments, a weight ratio of the fragrance is about 0.01 wt % to about 40 wt % relative to the formulation.

In some embodiments, the formulation further comprises a permeabilizer selected from polyethylenimine (PEI), lactic acid, or a combination thereof.

Advantageously, the permeabilizer improves the anti-microbial effect by making the microbial cell membrane more susceptible to the cashew testa extract and iron particle and/or iron oxide particle.

In some embodiments, a weight ratio of the permeabilizer is about 0.01 wt % to about 25 wt % relative to the formulation.

In some embodiments, the formulation further comprises a surfactant selected from a cocoamphoacetate salt, taurates, isethionates, olefin sulfonates, sulfosuccinates, sodium lauriminodipropionate, disodium lauroamphodiacetate, and polysorbate esters.

In some embodiments, the surfactant is decyl glucoside.

In some embodiments, a weight ratio of the surfactant is about 0.5 wt % to about 80 wt % relative to the formulation.

In some embodiments, the formulation further comprises a film forming agent selected from (3-glycidyloxypropyl)trimethoxysilane and/or gelatin.

In some embodiments, a weight ratio of the film forming agent is about 10 wt % to about 25 wt % relative to the formulation.

In some embodiments, the formulation further comprises a solvent selected from water, ethyl acetate, or a combination thereof.

In some embodiments, the formulation further comprises powdered cellulose.

In some embodiments, a weight ratio of the cellulose is about 1 wt % to about 20 wt % relative to the formulation.

In some embodiments, the powdered cellulose is extracted from a fruit rind and/or a symbiotic culture of bacteria and yeast (SCOBY).

In some embodiments, the formulation further comprises maltodextrin.

In some embodiments, the cashew testa extract comprises phenolic compounds selected from tannins, catechin, epicatechin, epigallocatechin, p-coumaric, gallic acid or a combination thereof.

In some embodiments, the iron particle and/or iron oxide particle is at least partially passivated by the phenolic compounds of the cashew testa extract.

In some embodiments, the cashew testa extract further comprises protein, amino acid, sugar, carbohydrate or a combination thereof.

In some embodiments, the iron particle and/or iron oxide particle are at least partially passivated by the protein, amino acid, sugar, carbohydrate or a combination thereof.

In some embodiments, the iron particle and/or iron oxide particle is a core-shell particle, the core is an elemental iron core or an iron alloy core, and the shell is an iron oxide shell.

In some embodiments, the cashew testa extract is at least partially incorporated in the shell of the iron particle and/or iron oxide particle.

In some embodiments, the formulation has an at least about 2 log reduction against E. coli after 5 min.

In some embodiments, the formulation has an at least about 2 log reduction against E. coli after 1 min.

In some embodiments, the formulation has an at least about 2 log reduction against S. aureus after 5 min.

In some embodiments, the formulation has an at least about 2 log reduction against S. aureus after 1 min.

In some embodiments, the formulation is for use as an antimicrobial coating, disinfectant, hand sanitizer, and/or soap.

In some embodiments, the formulation is applied on at least a surface of a fabric for use as a wet wipe.

In some embodiments, the fabric is a nonwoven fabric.

In some embodiments, the fabric comprises cellulose extracted from a fruit rind and/or a symbiotic culture of bacteria and yeast (SCOBY).

In some embodiments, the cellulose is extracted from durian rind.

The present invention also provides method of disinfecting a non-biological surface, comprising use of a formulation as disclosed herein.

The present invention also provides method of coating a non-biological surface with an anti-microbial coating, comprising use of a formulation as disclosed herein.

The present invention also provides method of sanitizing a biological surface, comprising use of a formulation as disclosed herein.

The present invention also provides method of providing an anti-microbial function to a textile, comprising use of a formulation as disclosed herein.

The present invention also provides a method of forming a formulation, comprising:
a) mixing a cashew testa extract, an iron particle and/or iron oxide particle and a carbohydrate;
wherein a weight ratio of the cashew testa extract to the iron particle and/or iron oxide particle is about 100:1 to about 1:200; and
wherein a weight ratio of the carbohydrate to the cashew testa extract and iron particle and/or iron oxide particle is about 30:1 to about 1:300; and
wherein the cashew testa extract at least partially incorporated in the iron particle and/or iron oxide particle.

In some embodiments, the method further comprises a step after step (a) of adjusting a pH of the natural ingredient formulation to of about 4 to about 5.

In some embodiments, the method further comprises a step after step (a) of adding an excipient selected from colorant, humectant, fragrance, stabilizer, permeabilizer, attachment promoter, film forming agent, transfection agent, surfactant, solvent, anti-oxidant or a combination thereof.

In some embodiments, the method further comprises a step after step (a) of diluting the formulation in an aqueous medium.

In some embodiments, the method further comprises a step before step (a) of reacting the cashew testa extract with an iron precursor in order to form the cashew testa extract and the iron particle and/or iron oxide particle.

### BRIEF DESCRIPTION OF THE DRAWINGS

Embodiments of the present invention will now be described, by way of non-limiting example, with reference to the drawings in which:

FIG. 1 is an exemplary schematic representation of the cashew testa extract composition;

FIG. 2 is an exemplary schematic representation of the cashew testa extract composition;

FIG. 3 shows Coomasie Brilliant Blue R dye degradation after exposure to cashew testa extract composition;

FIG. 4 shows Coomasie Brilliant Blue R dye degradation after exposure to cashew testa extract composition;

FIG. 5 shows OH radical measurement results after exposure to cashew testa extract composition;

FIG. 6 shows O₂ radical measurement results after exposure to cashew testa extract composition for 2 h; and

FIG. 7A-D shows Scanning Electron Microscope (SEM) images of iron particles and/or iron oxide particles with cashew testa extract incorporated.

### DETAILED DESCRIPTION

The inventor has envisioned that a formulation with anti-microbial (anti-bacterial and/or anti-viral) properties can be advantageous. The formulation can be applied or used on a surface as a disinfectant or on a textile. To this end, the inventor has found that an extract from natural product can be particularly advantageous. In particular, the inventors have found that a cashew testa extract has anti-microbial efficacy. When formulated as a formulation as disclosed herein, the anti-microbial efficacy can be synergistically improved. By using utilising and upcycling food waste and transform it into higher value products, food waste problems can also be reduced.

Without wanting to be bound by theory, it is believed that the cashew testa extract has the ability to kill viruses and bacteria on contact. When the extract is applied on a substrate, the compounds in the extract can kill viruses and bacteria which are on the surface of the substrate. It is postulated that the extract can trap the microbes by mimicking the sites on human cells to which they normally attach, and then destroy them by disrupting their surfaces (viruses) and cell walls (bacteria). It was found that the extract can kill germs which can cause Influenza A, Bird Flu, SARS, measles, pneumonia, common colds, tuberculosis, herpes, MRSA and gastroenteritis.

The anti-bacterial mechanism of action is believed to be the binding of the proteins on bacterial cell membranes to at least the phenolic compounds in the extract, and in doing so damages the bacterial cell's structure and function. Further complexation with essential metal ions also inhibits fibrin formation. Further, the cashew testa extract can also have anti-viral activity. It is believed that the compounds in the extract can target the different stages of the viral replication process. This includes the extracellular virions themselves, during the attachment of the virus to the cell, during the penetration of the virus into the cell, during the viral replication process in the host cell, as well during the assembly of new viral particles, transport proteins, polysaccharides, and viral enzymes. In almost all of the abovementioned stages, the composite will bind permanently to the proteins of the capsid or supercapsid. The proteins may be either the specific viral enzymes required for viral replication or to the newly synthesized viral proteins that are involved in the production of the new viral particle.

Towards this end, the inventors have found that the anti-microbial efficacy of the cashew testa extract can be increased if the cashew testa extract is coupled with iron particles and/or iron oxide particles. It is believed that the addition of iron particles and/or iron oxide particles provide a multi-defence mechanism against bacteria and viruses. The iron particles and/or iron oxide particles are a ROS producing active ingredient. The active ingredient generates ROS continuously, which diffuses free radicals which kills a broad spectrum of bacteria and viruses for an extended period of time.

Further advantageously, a natural polymer (such as carbohydrate) provides an instant contact-killing action on the microbes. In addition, it helps the active ingredient bind to surfaces, forming a positively charged “electric fence”, resulting in long term integrity and durability. To this end, when combined with a carbohydrate at particular ratios, it was found that the carbohydrate improves the retention time of the radicals produced by the cashew testa extract and iron particle and/or iron oxide particle on the applied surface. The combination of sustained diffuse and acute contact mechanisms warrants a highly effective antibacterial and antiviral performance.

As an example, a dual defence system against bacteria and viruses can be provided by spraying the formulation on a surface. After drying, a thin coating will form on the treated surface. As a first defence, radicals steadily diffuse to the surrounding (˜1 mm) and kill bacteria and viruses that are not in close proximity with the surface. The diffusion of radicals can takes place continuously over 3 months to provide long-lasting effectiveness. As a second defence, any bacteria or viruses which breaks through the first defence and comes into direct contact with the surface will have the cell membrane physically “tear up” which kills the microbe instantaneously. The integrated killing actions of sustained diffusion and acute contact warrants an effective antibacterial and antiviral performance.

Accordingly, the present invention provides a formulation, comprising:
a) cashew testa extract;
b) an iron particle and/or iron oxide particle; and
c) a carbohydrate;
wherein a weight ratio of the cashew testa extract to the iron particle and/or iron oxide particle is about 100:1 to about 1:200; and
wherein a weight ratio of the carbohydrate (c) to the cashew testa extract and iron particle and/or iron oxide particle (a and b) is about 8:1 to about 1:300.

In some embodiments, the formulation comprising:
a) cashew testa extract;
b) an iron particle and/or iron oxide particle; and
c) a carbohydrate;
wherein a weight ratio of the cashew testa extract to the iron particle and/or iron oxide particle is about 100:1 to about 1:200; and
wherein a weight ratio of the carbohydrate (c) to the cashew testa extract and iron particle and/or iron oxide particle (a and b) is about 30:1 to about 1:300.

In some embodiments, the formulation comprises:
a) cashew testa extract;
b) an iron particle and/or iron oxide particle; and
c) a carbohydrate;
wherein a weight ratio of the cashew testa extract to the iron particle and/or iron oxide particle is about 100:1 to about 1:200; and
wherein a weight ratio of the carbohydrate (c) to the cashew testa extract and iron particle and/or iron oxide particle (a and b) is about 8:1 to about 1:300; and
wherein the cashew testa extract is at least partially incorporated in the iron particle and/or iron oxide particle.

In some embodiments, the formulation comprises:
a) cashew testa extract;
b) an iron particle and/or iron oxide particle; and
c) a carbohydrate;
wherein a weight ratio of the cashew testa extract to the iron particle and/or iron oxide particle is about 100:1 to about 1:200; and
wherein a weight ratio of the carbohydrate (c) to the cashew testa extract and iron particle and/or iron oxide particle (a and b) is about 30:1 to about 1:300; and
wherein the cashew testa extract is at least partially incorporated in the iron particle and/or iron oxide particle.

In some embodiments, the formulation, comprises:
a) cashew testa extract;
b) an iron particle and/or iron oxide particle; and
c) a carbohydrate;
wherein a weight ratio of the cashew testa extract to the iron particle and/or iron oxide particle is about 100:1 to about 1:100; and
wherein a weight ratio of the carbohydrate (c) to the cashew testa extract and iron particle and/or iron oxide particle (a and b) is about 15:1 to about 15:14; and
wherein the cashew testa extract at least partially incorporated in the iron particle and/or iron oxide particle.

In some embodiments, the formulation, comprises:
a) cashew testa extract;
b) an iron particle and/or iron oxide particle; and
c) a carbohydrate;
wherein a weight ratio of the cashew testa extract to the iron particle and/or iron oxide particle is about 100:1 to about 1:100; and
wherein a weight ratio of the carbohydrate (c) to the cashew testa extract and iron particle and/or iron oxide particle (a and b) is about 8:1 to about 1:1; and
wherein the cashew testa extract at least partially incorporated in the iron particle and/or iron oxide particle.

Advantageously, the formulation does not rely on harsh chemicals which can be damaging to a surface or a user's hand. Towards this end, natural products were relied on for the anti-microbial effect as much as possible.

Cashew Testa Extract

Cashew nut (Anacardium occidentale) is a commonly consumed nut worldwide. The nut is covered by a reddish-brown skin, known as testa. The testa is commonly removed and discarded during processing due to its bitter and astringent taste. It is advantageous to use these discarded material to synthesis an anti-microbial composition as the production cost is low and it is also environmentally green and friendly.

Cashew testa extract contains many polyphenols, including tannic acid. Tannic acid is a water soluble and reddish brown coloured molecule. It is believed that tannic acid, and in general phenolic compounds, can have an anti-microbial effect by binding to the bacteria, disrupt the bacteria's cell membrane integrity and also disrupt various functions inside the bacteria cell.

One hypothesis is that changes in various intracellular functions induced by hydrogen binding of the phenolic compounds to enzymes or by the modification of the cell wall rigidity with integrity losses due to different interactions with the cell membrane. This may induce irreversible damages of the cytoplasmic membrane and coagulation of the cell content that can even lead to the inhibition of intracellular enzymes. For example, condensed phenylpropanoids-tannins may induce damages at the cell membrane and even inactivate the metabolism by binding to enzymes while phenolic acids have been shown to disrupt membrane integrity, as they cause consequent leakage of essential intracellular constituents. Flavonoids may link to soluble proteins located outside the cells and with bacteria cell walls thus promoting the formation of complexes. Flavonoids also may act through inhibiting both energy metabolism and DNA synthesis thus affecting protein and RNA syntheses. In the case of Gram-positive bacteria, intracellular pH modification as well as interference with the energy (ATP) generating system were reported.

In some embodiments, the cashew testa extract comprises polyphenols or phenolic compounds such as tannins, catechin, epicatechin, epigallocatechin, and p-coumaric, gallic acid, or a combination thereof. These polyphenols possess high antioxidant and demonstrate high free radical scavenging activity. The compounds of the phenolic compounds can be in any desired percentages or ratios. The phenolic compounds as used herein refer to chemical compounds that contain at least one aromatic ring with hydroxyl groups (—OH) attached.

In some embodiments, the cashew testa extract comprises protein, amino acid, sugar, carbohydrate or a combination thereof.

“Polypeptide”, “peptide” and “protein” are used interchangeably herein to refer to any polymer of amino acid residues (dipeptide or greater) linked through peptide bonds or modified peptide bonds and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. Polypeptides of the present invention include, but are not limited to, naturally purified products, products of chemical synthetic procedures, and products produced by recombinant techniques from a prokaryotic or eukaryotic host, including, for example, bacterial, yeast, higher plant, insect and mammalian cells. The polypeptides of the invention may comprise non-peptidic components, such as carbohydrate groups. Carbohydrates and other non-peptidic substituents may be added to a polypeptide by the cell in which the polypeptide is produced, and will vary with the type of cell. For polypeptides that are made recombinantly, the nature and extent of the modifications in large part will be determined by the post-translational modification capacity of the particular host cell and the modification signals that are present in the amino acid sequence of the polypeptide in question. For instance, glycosylation patterns vary between different types of host cell. Polypeptides are defined herein, in terms of their amino acid backbone structures; substituents such as carbohydrate groups are generally not specified, but may be present nonetheless. In addition, polypeptides of the invention may also include an initial modified methionine residue, in some cases as a result of host-mediated processes. Proteins may be present as monomeric or as multimeric proteins e.g. as dimers (homo or heterodimers) or trimers.

In the context of this specification, the term “amino acid” is defined as having at least one primary, secondary, tertiary or quaternary amino group, and at least one acid group, wherein the acid group may be a carboxylic, sulfonic, or phosphonic acid, or mixtures thereof. The amino groups may be “alpha”, “beta”, “gamma” . . . to “omega” with respect to the acid group(s). The backbone of the “amino acid” may be substituted with one or more groups selected from halogen, hydroxy, guanido, heterocyclic groups.

Thus term “amino acids” also includes within its scope glycine, alanine, valine, leucine, isoleucine, methionine, proline, phenylalanine, tryptophane, serine, threonine, cysteine, tyrosine, asparagine, glutamine, asparte, glutamine, lysine, arginine and histidine, taurine, betaine, N-methylalanine etc. (L) and (D) forms of amino acids are included in the scope of this invention.

In some embodiments, the cashew testa extract comprises:
a) phenolic compounds selected from tannins, catechin, epicatechin, epigallocatechin, p-coumaric, gallic acid or a combination thereof; and
b) protein, amino acid, sugar, carbohydrate or a combination thereof.

In some embodiments, a weight ratio of phenolic compounds to protein is about 1:100 to about 100:1. In other embodiments, the weight ratio is about 1:90 to about 100:1, about 1:80 to about 100:1, about 1:70 to about 100:1, about 1:60 to about 100:1, about 1:50 to about 100:1, about 1:40 to about 100:1, about 1:20 to about 100:1, about 1:10 to about 100:1, about 1:1 to about 100:1, about 10:1 to about 100:1, about 20:1 to about 100:1, about 30:1 to about 100:1, about 40:1 to about 100:1, about 50:1 to about 100:1, about 60:1 to about 100:1, or about 70:1 to about 100:1.

In some embodiments, a weight ratio of phenolic compounds to sugar is about 1:100 to about 100:1. In other embodiments, the weight ratio is about 1:90 to about 100:1, about 1:80 to about 100:1, about 1:70 to about 100:1, about 1:60 to about 100:1, about 1:50 to about 100:1, about 1:40 to about 100:1, about 1:20 to about 100:1, about 1:10 to about 100:1, about 1:1 to about 100:1, about 10:1 to about 100:1, about 20:1 to about 100:1, about 30:1 to about 100:1, about 40:1 to about 100:1, about 50:1 to about 100:1, about 60:1 to about 100:1, or about 70:1 to about 100:1.

Combination of Cashew Testa Extract and Iron and/or Iron Oxide Particles

The inventors have found that by combining cashew testa extract with iron particles and/or iron oxide particles, the anti-microbial effect can be further improved. For example, the retention time of the extract on the surface can be further improved. The compounds of the extract in the comparator are also easily degraded, and hence the anti-microbial efficacy falls off rapidly. It was also further found that when the comparator is applied to a porous film, the compounds of the comparator extract are only retained on the interface and do not penetrate. Accordingly, the anti-microbial effects are not long lasting.

Microbial infections and the development of antimicrobial resistance have received attention as one of the most critical issues facing the public health and security. The creation of clean antimicrobial surfaces with long-term stabilities and activities have tremendous applications involving almost all aspects of our daily life, such as medical devices, hospital surfaces, textiles, packaging, electrical appliances, marine antifouling, filters and public surfaces. Inorganic antimicrobial materials, especially semiconductor antimicrobial materials are less prone to chemical contamination and possess long-term stability. Some metal or metal oxides, such as silver, zinc oxide and titanium oxide particles have been used as antimicrobial ingredients in various products or in antimicrobial surface coatings. However, these materials also have limitations such as heavy metal contamination/toxicity (for Ag based materials). For ZnO and TiO₂ materials, they suffer from low antimicrobial efficacies and limited applications, due to the dependence on photo irradiation. In addition, uncertainty nano-toxicity is another concern for nano-size materials.

Towards this end, the inventors have found that the anti-microbial efficacy can be further improved synergistically (or at least additively) through the addition of iron particles and/or iron oxide particles. Advantageously, the inventors have found that an iron based antimicrobial material is non-toxic, yet can be highly active against microbes, very stable and has long-term activity. For example, the iron-iron oxide composition can be synthesized by modifying iron powder (microns size) with carbohydrates, amino acids, food additives or nutrition, under non organic solvent conditions. This can be done, for example, using a fluidised bed reactor or a sonicator.

Iron powder is redox active, it will slowly react with oxygen and moisture to form iron oxides and releasing hydrogen. Iron powder by itself will not generate reactive oxygen species (ROS) and does not kill bacteria. There could be some iron cation releasing out from iron powder, but it is in very low concentration and not harmful to cells.

The iron particles can have a nano-structured protection shell covered the iron core. This shell can, for example, be an iron oxide shell. The core-shell structure creates a special interface between iron core and iron complex shell which will change the potential of iron core and change the redox reaction pathway. Towards this end, it is believed that this self-corrosion process could also happen on Fe/FeₑO₃, Fe/FeₑO₃ particles and/or a combination thereof. The electrons generated from iron corrosion can be transferred into the conduction band (CB) of iron oxide in an energetically favourable way. The electrons in the CB are able to reduce oxygen and generate ROS. The iron particles could generate different ROS which include super oxide, singlet oxygen and hydroxyl radical. In other words, the electrons are donated from iron to iron oxide (CB) and reduce oxygen molecules to generate radicals in an energetically favorable way. The whole system does not rely on an external stimulus and the ROS generation process could be manipulated and have long-term stability. ROS will then kill contacted bacteria and virus. The ROS killing mechanism of the material while being similar to photo catalyst materials, such as ZnO and TiO₂, defers in that the iron/iron oxide particles is a self-catalysed material, it does not rely on photo irradiation to generate ROS. The iron particles sacrifices its iron core to generate ROS. New materials designed based on this concept could play pivotal roles as non-toxic and safe antimicrobial technology to replace organic disinfectants, antiseptics and antibiotics in a broad range of applications, especially in the control of infectious disease and antimicrobial resistance (AMR) transmission.

As used herein, ‘particles’ refer to micron-sized particles and/or nano-sized particles. Microparticles are particles between 1 and 1000 μm in size. Nanoparticles are particles between 1 nm to 1000 nm in size. The particles can be of any shape or morphology, such as spherical, rod-like, or asymmetrical.

In some embodiments, the iron particle and/or iron oxide particle is provided as a powder with a particle size of about 10 nm to about 500 μm. The iron particles and/or iron oxide particles can be micron sized particles. In other embodiments, the particle size is about 1 μm to about 450 μm, about 1 μm to about 400 μm, about 1 μm to about 350 μm, about 1 μm to about 300 μm, about 1 μm to about 250 μm, about 1 μm to about 200 μm, about 1 μm to about 150 μm, about 1 μm to about 100 μm, about 1 μm to about 90 μm, about 1 μm to about 80 μm, about 1 μm to about 70 μm, about 1 μm to about 60 μm, about 1 μm to about 50 μm, about 1 μm to about 50 μm, about 1 μm to about 40 μm, or about 10 μm to about 40 μm. The iron particles and/or iron oxide particles can be nano sized particles. In other embodiments, the particle size is about 10 nm to about 900 nm, about 10 nm to about 800 nm, about 10 nm to about 700 nm, about 10 nm to about 600 nm, about 10 nm to about 500 nm, about 10 nm to about 400 nm, about 10 nm to about 300 nm, about 10 nm to about 200 nm, about 10 nm to about 100 nm, about 10 nm to about 80 nm, about 10 nm to about 60 nm, or about 10 nm to about 40 nm.

In some embodiments, iron particle is used. In other embodiments, iron oxide particle is used. In this context, the distribution of iron or iron oxide is homogenous throughout the particle. In some embodiments, the iron particle and/or iron oxide particle is a core-shell particle. For example, the core can be an elemental iron core or an iron alloy core. The shell can be an iron oxide shell. In some embodiments, the iron particles and/or iron oxide particles is an iron-iron oxide particle. In some embodiments, the iron-iron oxide composition comprises iron, iron (II) oxide and iron (III) oxide. In some embodiments, the iron relative to the iron-iron oxide composition is at more than 90% w/w. In other embodiments, the iron relative to the iron-iron oxide composition is at more than 91% w/w, more than 92% w/w, more than 93% w/w, more than 94% w/w, more than 95% w/w, more than 96% w/w, or more than 97% w/w. In other embodiments, the iron relative to the iron-iron oxide composition is at more than 15% w/w, more than 20% w/w, more than 25% w/w, more than 30% w/w, more than 35% w/w, more than 40% w/w, more than 45% w/w, more than 50% w/w, more than 55% w/w, more than 60% w/w, more than 65% w/w, more than 70% w/w, or more than 75% w/w. In other embodiments, the iron (II) oxide and iron (III) oxide relative to the iron-iron oxide composition is at less than 10% w/w. In other embodiments, the iron (II) oxide and iron (III) oxide relative to the iron-iron oxide composition is at less than 9% w/w, at less than 8% w/w, at less than 7% w/w, at less than 6% w/w, at less than 5% w/w, at less than 4% w/w, or at less than 3% w/w. In other embodiments, the iron (II) oxide and iron (III) oxide relative to the iron-iron oxide composition is at less than 45% w/w, less than 40% w/w, less than 35% w/w, less than 30% w/w, less than 25% w/w, less than 20% w/w, less than 15% w/w, less than 10% w/w %, or less than 5% w/w. The relative weight ratios can, for example, be determined using Energy-dispersive X-ray Spectroscopy (EDX).

In some embodiments, the iron is elemental iron. In other embodiments, the iron (II) oxide is FeO. In other embodiments, the iron (II) oxide is Fe₂O₃. In other embodiments, the iron (II) oxide and iron (III) oxide is Fe₃O₄. In this regard, the iron-iron oxide composition can be a mixture of Fe, FeO, Fe₂O₃ and Fe₃O₄.

In some embodiments, the iron particle and/or iron oxide particle comprises elemental Fe, FeO, Fe₂O₃, Fe₃O₄, or a combination thereof.

In some embodiments, the cashew testa extract and the iron particle and/or iron oxide particle are physically mixed. In this regard, components of the cashew testa extract can be at least partially incorporated in the iron particle and/or iron oxide particle by forming a shell which encapsulates the iron particle and/or iron oxide particle. Alternatively, the cashew testa extract can at least partially passivates the surface of the iron particle and/or iron oxide particle in order to form a cashew-iron particle. For example, the particles can have an iron core, which can be encapsulated with a cashew testa extract shell. In some embodiments, the shell comprises phenolic compound, amino acid, carbohydrate or a mixture thereof. In some embodiments, the shell comprises an amino acid. In some embodiments, the shell comprises a carbohydrate. In some embodiments, the shell comprises a phenolic compound.

Advantageously, it was found that the physical combination of cashew testa extract with iron particles and/or iron oxide particles act synergistically (or at least additively) on each other such that the anti-microbial effect is increased. Without wanting to be bound by theory, it is believed that when the compounds in cashew testa extract are allowed to passivate the iron/iron oxide particles to form the cashew-iron particles, aggregation and/or agglomeration of iron/iron oxide particles can be decreased. Further, as the stability of the particles are improved, it was found that when applied to a surface, the particles can reside on the surface without rapid degradation. This is in contrast to cashew testa extract alone (which tends to degrade rapidly) or iron/iron oxide particles alone (which are not retained on the surface). Accordingly, the retention time of the formulation on the surface is improved, and the anti-microbial efficacy is correspondingly increased.

Further, when at least the phenolic compounds adsorb on the surface of the iron/iron oxide particles, the particles were found to be slightly protected from degraded, hence ensuring that the anti-microbial effect is longer lasting.

In some embodiments, the iron particles and/or iron oxide particles are at least partially passivating by the cashew testa extract to form the cashew-iron particles. In some embodiments, the iron oxide particles is at least partially passivated by the phenolic compounds in the cashew testa extract. Towards this end, the cashew testa extract can physically adsorb on the surface of the iron oxide particles through like-like interactions. This provides for greater stability of the composition, and hence shelf-life.

In some embodiments, the iron oxide particles is at least partially passivated by the sugar and/or carbohydrate in the cashew testa extract. In other embodiments, the iron particles and/or iron oxide particles further comprises a carbohydrate. In some embodiments, the carbohydrate relative to the iron-iron oxide composition is at about 2% w/w to about 6% w/w. In other embodiments, the carbohydrate relative to the iron-iron oxide composition is at about 2% w/w to about 5% w/w, or about 3% w/w to about 5% w/w. In other embodiments, the carbohydrate relative to the iron-iron oxide composition is at about 20% w/w to about 60% w/w, about 20% w/w to about 55% w/w, about 20% w/w to about 50% w/w, about 20% w/w to about 45% w/w, about 20% w/w to about 40% w/w, about 20% w/w to about 35% w/w, or about 20% w/w to about 30% w/w. The relative weight ratios can, for example, be determined using Energy-dispersive X-ray Spectroscopy (EDX).

The carbohydrate can be selected from monosaccharide, disaccharide, oligosaccharide, and polysaccharide. Examples of carbohydrate are, but not limited to, glucose, galactose, fructose, xylose, sucrose, lactose, maltose, trehalose, sorbitol, mannitol, maltodextrin, raffinose, stachyose, fructo-oligosaccharide, amylose, amylopectin, modified starch, glycogen, dextran, chitosan, glycosaminoglycans, alginate, ulvan, gum Arabic, gellan gum, cellulose, hemicellulose, ethylcellulose, methylcellulose, pectin, hydrocolloid and a combination thereof.

In some embodiments, the iron oxide particles is at least partially passivated by the amino acid in the cashew testa extract. In other embodiments, the iron particles and/or iron oxide particles further comprises an amino acid. In some embodiments, the amino acid relative to the iron-iron oxide composition is at about 2% w/w to about 6% w/w. In other embodiments, the amino acid relative to the iron-iron oxide composition is at about 2% w/w to about 5% w/w, or about 3% w/w to about 5% w/w.

In some embodiments, the iron oxide particles is further at least partially passivated by a carboxylate moiety or a hydroxyl moiety. In some embodiments, the carboxylic acid is selected from fatty acid, aromatic carboxylic acid, dicarboxylic acid, tricarboxylic acid, keto acid, α-hydroxyl acid, divinylether fatty acid, phosphoric acid, polyphosphoric acid, tungstic acid, vanadic acid, molybdic acid, heteropoly acid, or a combination thereof. In some embodiments, the carboxylic acid is selected from benzoic acid, phosphoric acid, sulphuric acid, or a combination thereof.

In some embodiments, a volume ratio or weight ratio of the cashew testa extract to iron particle and/or iron oxide particle is about 100:1 to about 1:200. In other embodiments, the ratio is about 100:1 to about 1:180, about 100:1 to about 1:160, about 100:1 to about 1:140, about 100:1 to about 1:120, about 100:1 to about 1:100, about 90:1 to about 1:90, 80:1 to about 1:80, 70:1 to about 1:70, 60:1 to about 1:60, 50:1 to about 1:50, 40:1 to about 1:40, 30:1 to about 1:30, 20:1 to about 1:20, 10:1 to about 1:10, or about 10:9, about 10:8, about 10:7, about 10:6, about 10:5, about 10:4, about 10:3, or about 10:2. In other embodiments, the weight ratio is about 90:1 to about 1:200, about 80:1 to about 1:200, about 70:1 to about 1:200, about 60:1 to about 1:200, about 50:1 to about 1:200, about 40:1 to about 1:200, about 30:1 to about 1:200, about 20:1 to about 1:200, about 10:1 to about 1:200, about 1:1 to about 1:200, about 90:1 to about 1:100, about 90:1 to about 1:90, about 80:1 to about 1:90, about 80:1 to about 1:80, about 70:1 to about 1:80, about 70:1 to about 1:70, about 60:1 to about 1:70, about 60:1 to about 1:60, about 50:1 to about 1:60, about 50:1 to about 1:50, about 40:1 to about 1:50, about 40:1 to about 1:40, about 30:1 to about 1:40, about 30:1 to about 1:30, about 20:1 to about 1:30, about 20:1 to about 1:20, about 10:1 to about 1:20, about 10:1 to about 1:10, about 10:1 to about 1:8, about 10:1 to about 1:6, about 10:1 to about 1:4, about 10:1 to about 1:2, or about 10:1 to about 1:1.

Alternatively, the iron particle and/or iron oxide particle can be chemically synthesised in situ, in the presence of an iron precursor and cashew testa extract. In some embodiments, the iron particles and/or iron oxide particles are formed in situ, by mixing an iron precursor with the cashew testa extract. As is shown in the examples, the iron particle precursor can be elemental iron powder, iron (III) salts, or a combination thereof.

In some embodiments, an anion of the iron (III) salt is selected from nitrate, chloride, bromide, fluoride, iodide, sulphate, oxalate, perchlorate, phosphate, tetrafluoroborate, or a combination thereof. The iron (III) salt can be its hydrated form thereof.

By forming the iron particles and/or iron oxide particles in situ, the cashew testa extract can be incorporated into the iron particles and/or iron oxide particles. For example, the cashew testa extract can be incorporated into the core or the shell of the iron particles and/or iron oxide particles. This advantageously provides greater stability to the iron particle and/or iron oxide particle against aggregation and agglomeration.

For example, the cashew testa extract can react with an iron powder (iron precursor) in order to form iron particle and/or iron oxide particle. Advantageously, by allowing the cashew testa extract to react with the iron powder, an activated shell of iron oxide and cashew testa extract can be formed on a core of elemental iron or iron alloy. The thickness of the iron oxide shell can be controlled by the reaction conditions as well as the amount of cashew testa extract to iron powder ratio. The formation of a cashew-iron oxide shell provides for a greater anti-microbial efficacy as the retention time is longer and the surface area of contact is increased. Additionally, it was found that having a core of elemental iron or iron alloy is advantageous as it regenerates the outer iron oxide shell as the shell gets ‘used up’.

In some embodiments, the cashew testa extract first react with an iron powder (iron precursor), followed by iron (III) salt (iron precursor) in order to form iron particle and/or iron oxide particle. This provides an iron particle and/or iron oxide particle with a core comprising elemental iron and the cashew testa extract, and a shell formed by iron (III) salt. Alternatively, iron particle and/or iron oxide particle can have an elemental iron core and a biphasic (layered) shell comprising the cashew testa extract and iron (III).

In other embodiments, the cashew testa extract first react with an iron (III) salt (iron precursor), followed by iron powder (iron precursor) in order to form iron particle and/or iron oxide particle. This provides iron particle and/or iron oxide particle can have an elemental iron core and a monophasic shell comprising a mixture of cashew testa extract and iron (III).

In some embodiments, the cashew testa extract react with an iron (III) salt (iron precursor) and iron powder (iron precursor) simultaneously in order to form iron particle and/or iron oxide particle. This provides an iron particle and/or iron oxide particle with a core comprising elemental iron and the cashew testa extract, and a shell comprising the cashew testa extract and iron (III). Advantageously, this allows for the formation of an activated shell of iron oxide and cashew testa extract on the core of an activated elemental iron or iron alloy. The thickness of the iron oxide shell can be controlled by the reaction conditions as well as the amount of cashew testa extract to iron precursor ratio. The formation of a cashew-iron oxide shell provides for a greater anti-microbial efficacy as the retention time is longer and the surface area of contact is increased. Additionally, it was found that having a core of elemental iron or iron alloy is advantageous as it regenerates the outer iron oxide shell as the shell gets ‘used up’.

Accordingly, in some embodiments, the iron particles and/or iron oxide particles comprises iron, Fe₃O₄, and phenolic compound, amino acid, carbohydrate or a mixture thereof. In some embodiments, the iron relative to the iron-iron oxide composition is at more than 95% w/w, the Fe₃O₄ relative to the iron-iron oxide composition is at less than 2% w/w, and the phenolic compound, amino acid, carbohydrate or a mixture thereof relative to the iron-iron oxide composition is at about 3% w/w to about 5% w/w.

In some embodiments, the iron particles and/or iron oxide particles comprises iron, Fe₃O₄ and a carbohydrate. In some embodiments, the iron relative to the iron-iron oxide composition is at more than 95% w/w, the Fe₃O₄ relative to the iron-iron oxide composition is at less than 2% w/w, and the carbohydrate relative to the iron-iron oxide composition is at about 3% w/w to about 5% w/w.

In some embodiments, the iron particles and/or iron oxide particles comprises iron, Fe₃O₄ and an amino acid. In some embodiments, the iron relative to the iron-iron oxide composition is at more than 95% w/w, the Fe₃O₄ relative to the iron-iron oxide composition is at less than 2% w/w, and the amino acid relative to the iron-iron oxide composition is at about 3% w/w to about 5% w/w. In some embodiments, the amino acid is selected from glycine, alanine, valine, leucine, isoleucine, methionine, proline, phenylalanine, tryptophane, serine, threonine, cysteine, tyrosine, asparagine, aspartic acid, glutamine, glutamic acid, lysine, arginine, histidine, taurine, betaine, N-methylalanine or a combination thereof.

In other embodiments, the iron particle and/or iron oxide particle further comprises an amino acid, carbohydrate or a mixture thereof. In some embodiments, the amino acid, carbohydrate or a mixture thereof relative to the iron-iron oxide composition is at about 2% w/w to about 6% w/w. In other embodiments, the amino acid, carbohydrate or a mixture thereof relative to the iron-iron oxide composition is at about 2% w/w to about 5% w/w, or about 3% w/w to about 5% w/w. For example, a mixture of methylcellulose and zein can be used.

In some embodiments, the iron particle and/or iron oxide particle is an iron core-shell particles, wherein the core comprises Fe and the shell comprises an amino acid. In other embodiments, the iron particles and/or iron oxide particles is a plurality of iron core-shell particles, wherein the core comprises Fe, the shell comprises an amino acid, and an interface between the core and the shell comprises iron oxide. In some embodiments, the iron oxide is Fe₃O₄.

In some embodiments, the iron particle and/or iron oxide particle is an iron core-shell particles, wherein the core comprises Fe and the shell comprises Fe and an amino acid. In other embodiments, the iron particle and/or iron oxide particle is an iron core-shell particles, wherein the core comprises Fe, the shell comprises Fe and an amino acid, and an interface between the core and the shell comprises iron oxide. In some embodiments, the iron oxide is Fe₃O₄.

Advantageously, when the iron particles are encapsulated with a shell, the iron core can be protected by an encapsulation shell material comprising the amino acid and/or carbohydrate. This may further delays the formation of iron oxide and hence the production of ROS until its use in the face mask. This prevents or reduces over oxidation of iron particles, and thus allows for a longer shelf life of the face mask and/or create a further persistence ROS over a longer period of time. Further, the amino acid and/or carbohydrate encapsulation material can change the potential of iron core and change the redox reaction pathway. For example, the iron oxide generated can form an interface layer between the shell and the Fe core. This allows the generation and release of ROS to be controlled. In this way, the shell on the iron particles controls the rate of iron oxidation, such that a constant release of ROS is available, which is sufficient for the anti-bacterial and/or anti-viral effect. This improves its suitability for multiple uses, allowing for additional washing. A further advantage is that as a natural compound such as a biopolymer is used to encapsulate the iron particle, the biodegradable nature of the biopolymer causes the shell to break down over time. For example, the shell can be broken down over time after multiple washing. This provides an additional anti-microbial and/or anti-viral persistence effect, as the previously less accessible inner iron core can now be more easily accessed.

In some embodiments, the shell has a thickness of about 5 nm to about 1 μm, or about 50 nm to about 400 nm. In other embodiments, the thickness is of about 50 nm to 350 nm, about 50 nm to about 300 nm, about 100 nm to about 300 nm, about 150 nm to about 300 nm, or about 200 nm to about 300 nm.

The shell can further comprise iron. In this regard, in some embodiments, the shell comprises iron and an amino acid, carbohydrate or a mixture thereof. Advantageously, as the iron is closer to the surface of the particles, the presence of iron in the shell can ‘kick starts’ the oxidation of iron to iron oxide. In this sense, a burst release of ROS is provided at first instance, which can provide protection to a user when the face mask is first used and before sufficient water is provided as moisture to the iron core.

In some embodiments, a volume ratio or weight ratio of the cashew testa extract to iron precursor is about 100:1 to about 1:100. In other embodiments, the ratio is about 90:1 to about 1:90, 80:1 to about 1:80, 70:1 to about 1:70, 60:1 to about 1:60, about 50:1 to about 1:50, about 40:1 to about 1:40, about 30:1 to about 1:30, about 20:1 to about 1:20, about 10:1 to about 1:10, or about 10:9, about 10:8, about 10:7, about 10:6, about 10:5, about 10:4, about 10:3, or about 10:2. In other embodiments, the ratio is about 1:1 to about 1:100, about 1:10 to about 1:100, about 1:20 to about 1:100, about 1:30 to about 1:100, about 1:40 to about 1:100, about 1:50 to about 1:100, about 1:60 to about 1:100, about 1:70 to about 1:100, about 1:80 to about 1:100, or about 1:90 to about 1:100. In other embodiments, the ratio is about 100:1 to about 1:1, about 100:1 to about 10:1, about 100:1 to about 20:1, about 100:1 to about 30:1, about 100:1 to about 40:1, about 100:1 to about 50:1, about 100:1 to about 60:1, about 100:1 to about 70:1, about 100:1 to about 80:1, or about 100:1 to about 90:1.

In some embodiments, the weight ratio of cashew testa extract to an iron content in the iron precursor is about 100:1 to about 1:100. In other embodiments, the ratio is about 90:1 to about 1:90, 80:1 to about 1:80, 70:1 to about 1:70, 60:1 to about 1:60, about 50:1 to about 1:50, about 40:1 to about 1:40, about 30:1 to about 1:30, about 20:1 to about 1:20, about 10:1 to about 1:10, or about 10:9, about 10:8, about 10:7, about 10:6, about 10:5, about 10:4, about 10:3, or about 10:2. In other embodiments, the ratio is about 1:1 to about 1:100, about 1:10 to about 1:100, about 1:20 to about 1:100, about 1:30 to about 1:100, about 1:40 to about 1:100, about 1:50 to about 1:100, about 1:60 to about 1:100, about 1:70 to about 1:100, about 1:80 to about 1:100, or about 1:90 to about 1:100. In other embodiments, the ratio is about 100:1 to about 1:1, about 100:1 to about 10:1, about 100:1 to about 20:1, about 100:1 to about 30:1, about 100:1 to about 40:1, about 100:1 to about 50:1, about 100:1 to about 60:1, about 100:1 to about 70:1, about 100:1 to about 80:1, or about 100:1 to about 90:1.

In some embodiments, the iron powder has an average particle size of about 10 nm to about 100 μm.

In some embodiments, the iron particle and/or iron oxide particle is an iron oxide nanoparticle. Iron oxide nanoparticles can be synthesised through plant-mediated green chemistry approach via using plant extract as a reducing agent and a metal precursor under suitable conditions. This process consists of three steps: (1) the activation phase in which the metal ions are reduced by the phenolic compounds in the plant extract followed by the nucleation of reduced metal atoms; (2) the growth phase, where small NPs adhere to form large sized NPs (Ostwald ripening); and (3) the termination phase, during which NPs attain their shape. The phenolic compounds can also act as stabilizing agents, capping the surface of the nanoparticles.

The iron oxide nanoparticle can be provided to the cashew testa extract for physical mixing. Alternatively, in other embodiments, the iron oxide nanoparticle is chemically synthesised in situ in the presence of iron precursor and cashew testa extract. The iron precursor can be iron (III) salt.

Advantageously, by allowing the cashew testa extract to react with the iron precursor, an activated iron oxide and cashew testa extract nanoparticle can be formed. In this method, the whole of the nanoparticle is activated. The size of the iron oxide nanoparticles can be controlled by the reaction conditions as well as the amount of cashew testa extract to iron oxide precursor ratio. Activity is found to be greater for these nanoparticles due to the increased surface area and surface energy, which favours an equilibrium towards dissolution.

In some embodiments, the iron oxide nanoparticles comprises elemental iron. In other embodiments, the iron oxide nanoparticles comprises an iron-cashew testa extract complex. In other embodiments, the iron oxide nanoparticles comprises an iron-phenolic compound complex. To this end, the nanoparticle is made up of network or matrix of iron atoms and phenolic compounds (or at least carbon atoms).

The iron oxide nanoparticles can be stabilised by the cashew testa extract. In some embodiments, the iron oxide nanoparticles are at least partially passivating by the phenolic compounds. Towards this end, the unreacted cashew testa extract can physically adsorb on the surface of the iron oxide nanoparticles through like-like interactions. This provides for greater stability of the composition, and hence shelf-life.

In some embodiments, the iron oxide nanoparticles has an average size of about 1 nm to about 1000 nm. In other embodiments, the average size is about 10 nm to about 50 nm.

As another example, iron oxide nanoparticles can be reacted with an iron powder in order to form iron oxide particles.

In both the physical mixing and chemical reaction of cashew testa extract composition with iron particles and/or iron oxide particles, the methods can further comprises a step of adding a base. The base such as NaOH, KOH, NH₄OH can be added to the iron particles and/or iron oxide particles. The addition of a base can, for example, facilitate the formation of iron oxide on the iron particles through an oxidation process.

The base can also be added to the iron oxide particle precursor. As Fe(OH)₂ and Fe(OH)₃ can formed at pH >8 by the hydroxylation of the ferrous and ferric ions, the formation of iron oxide particles can be facilitated.

The base can also be added to the cashew testa extract composition. For example, the oxidation of phenolic compounds can be controlled by the amount of NaOH added into the reaction. This can drive the formation of the iron oxide particle as well as the adsorption of the phenolic compounds for passivating the surfaces. Further, by controlling the ionisation of the phenolic compounds in the cashew testa extract composition through controlling the pH, the anti-microbial efficacy can be varied.

For example, the sequential addition of the reagents for reaction can be:

| Example | 1^{st} reagent | 2^{nd} reagent | 3^{rd} reagent |
|---|---|---|---|
| A | Cashew testa | Iron powder | |
| | extract | | |
| B | Cashew testa | iron oxide particle | |
| | extract | precursor | |
| C | Cashew testa | Iron powder | iron oxide particle |
| | extract | | precursor |
| D | Cashew testa | iron oxide particle | Iron powder |
| | extract | precursor | |
| E | Cashew testa | Iron powder and iron | |
| | extract | oxide particle precursor | |

In some embodiments, the weight ratio of the cashew testa extract and iron particle and/or iron oxide particle (a and b) is about 0.5 wt % to about 8 wt % relative to the formulation. In some embodiments, the weight ratio is about 1 wt % to about 8 wt %, about 1 wt % to about 7 wt %, about 1 wt % to about 6 wt %, about 1 wt % to about 5 wt %, or about 1 wt % to about 4 wt %.

Combination of Cashew Testa Extract. Iron Particle and/or Iron Oxide Particle and Carbohydrate

The natural ingredient formulation comprises a carbohydrate.

As used herein, a carbohydrate is a biomolecule consisting of carbon (C), hydrogen (H) and oxygen (O) atoms, usually with a hydrogen-oxygen atom ratio of 2:1 (as in water) and thus with the empirical formula Cₘ(H₂O)ₙ (where m may be different from n). However, not all carbohydrates conform to this precise stoichiometric definition (e.g., uronic acids, deoxy-sugars such as fucose). The term is a synonym of saccharide, a group that includes sugars, starch, and cellulose. The saccharides are divided into four chemical groups: monosaccharides, disaccharides, oligosaccharides, and polysaccharides. Monosaccharides and disaccharides, the smallest (lower molecular weight) carbohydrates, are commonly also referred to as sugars. Examples of carbohydrates or sugars are monosaccharides such as glucose, galactose, fructose, xylose; disaccharides such as sucrose, lactose, maltose, trehalose, polyols such as sorbitol, mannitol; oligosaccharides such as malto-oligosaccharides (maltodextrins), raffinose, stachyose, fructo-oligosaccharides; polysaccharides such as starch (amylose, amylopectin, modified starches) and non-starch polysaccharides (glycogen, cellulose, hemicellulose, pectins, hydrocolloids).

The weight ratio of the carbohydrate (c) to the cashew testa extract and iron particle and/or iron oxide particle (a and b) is about 8:1 to about 1:300. In other embodiments, the weight ratio is about 8:1 to about 1:250, about 8:1 to about 1:200, about 8:1 to about 1:150, about 8:1 to about 1:100, about 8:1 to about 1:50, about 8:1 to about 1:10, about 8:1 to about 1:5, about 8:1 to about 1:1, about 6:1 to about 1:1, or about 5:1 to about 1:1. In other embodiments, the weight ratio is about 5:1, about 4:1, about 3:1, about 2:1, or about 1:1. In other embodiments, the weight ratio is about 30:1 to about 1:300, about 30:1 to about 1:280, about 30:1 to about 1:260, about 30:1 to about 1:240, about 30:1 to about 1:220, about 30:1 to about 1:200, about 30:1 to about 1:180, about 30:1 to about 1:160, about 30:1 to about 1:140, about 30:1 to about 1:120, about 30:1 to about 1:100, about 30:1 to about 1:90, about 30:1 to about 1:80, about 30:1 to about 1:70, about 30:1 to about 1:60, about 30:1 to about 1:50, about 30:1 to about 1:40, about 30:1 to about 1:30, about 30:1 to about 1:25, about 30:1 to about 1:20, about 25:1 to about 1:30, about 20:1 to about 1:30, about 20:1 to about 1:20, about 15:1 to about 15:12, about 15:1 to about 15:10, about 15:1 to about 15:8, about 15:1 to about 15:6, about 15:1 to about 15:4, or about 15:1 to about 15:2.

Advantageously, when formulated in this ratio, the carbohydrate improves the retention time of the radicals produced by the cashew testa extract and iron particle and/or iron oxide particle on the applied surface. Further, the carbohydrate improves the adherence of the cashew testa extract and iron particle and/or iron oxide particle on applied surface. This serves to further improve the anti-microbial effect of the cashew testa extract and iron particle and/or iron oxide particle.

In some embodiments, the weight ratio of the carbohydrate is about 1 wt % to about 15 wt % relative to the formulation. In other embodiments, the weight ratio is about 2 wt % to about 15 wt %, about 3 wt % to about 15 wt %, about 4 wt % to about 15 wt %, about 5 wt % to about 15 wt %, about 5 wt % to about 14 wt %, about 5 wt % to about 13 wt %, about 5 wt % to about 12 wt %, about 5 wt % to about 11 wt %, or about 5 wt % to about 10 wt %.

In some embodiments, the carbohydrate is selected from chitosan, malto-oligosaccharides (maltodextrins), raffinose, stachyose, fructo-oligosaccharides; polysaccharides such as starch (amylose, amylopectin, modified starches) and non-starch polysaccharides (glycogen, cellulose, hemicellulose, pectins, hydrocolloids). In other embodiments, the carbohydrate is chitosan.

Chitosan is a linear polysaccharide composed of randomly distributed 0-(1-4)-linked D-glucosamine (deacetylated unit) and N-acetyl-D-glucosamine (acetylated unit). It can be made by treating the chitin shells of shrimp and other crustaceans with an alkaline substance, such as sodium hydroxide.

In some embodiments, chitosan has a molecular weight of about 2 kDa to about 500 kDa. In other embodiments, the chitosan has a molecular weight of about 2 kDa to about 400 kDa, about 2 kDa to about 350 kDa, about 2 kDa to about 300 kDa, about 2 kDa to about 250 kDa, about 2 kDa to about 200 kDa, about 2 kDa to about 150 kDa, about 2 kDa to about 100 kDa, about 2 kDa to about 80 kDa, or about 2 kDa to about 50 kDa. In other embodiments, chitosan has a molecular weight of about 10 kDa to about 300 kDa, about 10 kDa to about 250 kDa, about 10 kDa to about 240 kDa, about 10 kDa to about 230 kDa, about 10 kDa to about 220 kDa, about 10 kDa to about 210 kDa, about 10 kDa to about 200 kDa, about 20 kDa to about 200 kDa, about 30 kDa to about 200 kDa, about 40 kDa to about 200 kDa, about 50 kDa to about 200 kDa, about 50 kDa to about 190 kDa, about 60 kDa to about 190 kDa, about 70 kDa to about 190 kDa, about 80 kDa to about 190 kDa, about 90 kDa to about 190 kDa, about 100 kDa to about 190 kDa, about 100 kDa to about 180 kDa, about 100 kDa to about 170 kDa, about 100 kDa to about 160 kDa, about 100 kDa to about 150 kDa, or about 100 kDa to about 140 kDa.

In some embodiments, chitosan has a viscosity of about 100 cP to about 1500 cP. In other embodiments, chitosan has a viscosity of about 100 cP to about 1400 cP, about 100 cP to about 1300 cP, about 100 cP to about 1200 cP, about 100 cP to about 1100 cP, about 100 cP to about 1000 cP, about 200 cP to about 1000 cP, about 200 cP to about 900 cP, about 300 cP to about 900 cP, about 400 cP to about 900 cP, about 400 cP to about 800 cP, about 400 cP to about 700 cP, or about 400 cP to about 600 cP.

Other Excipients

In some embodiments, the natural ingredient formulation further comprises an excipient.

“Excipients” are inactive substances that serve as the vehicle or medium for an active substance, and include any and all solvents, dispersion media, inert diluents, or other liquid vehicles, dispersion or suspension aids, granulating agents, surface active agents, disintegrating agents, isotonic agents, thickening or emulsifying agents, preservatives, binding agents, lubricants, buffering agents, oils, and the like. Various excipients used in formulating compositions and known techniques for the preparation thereof is disclosed in G. A. R. Remington: The Science and Practice of Pharmacy, 21st ed. (2006), Lippincott Williams & Wilkins. Except insofar as any conventional excipient is incompatible with a substance or its derivatives, such as by producing any undesirable biological effect or otherwise interacting in a deleterious manner with any other component(s) of the pharmaceutical composition, its use is contemplated to be within the scope of this invention.

Excipients such as colouring agents, coating agents, anti-oxidants or preservatives and perfuming agents can be present in the composition, according to the judgment of the formulator. Examples of excipients are colloidal silica, hydroxypropyl methylcellulose, vitamin A, vitamin E, vitamin C, retinyl palmitate, selenium, sodium metabisulphite, propyl gallate, cysteine, methionine, citric acid, sodium citrate, methyl paraben, propyl paraben, benzalkaniumchloride, and lanolin.

Advantageously, this allows for the ease of transport and storage of the composition. The shelf-life of the composition can also be further improved.

In some embodiments, the excipient is selected from colorant, humectant, fragrance, stabilizer, permeabilizer, attachment promoter, film forming agent, transfection agent, surfactant, solvent, anti-oxidant or a combination thereof.

In some embodiments, the natural ingredient formulation further comprises a colorant selected from a terpene family. In other embodiments, the colorant is selected from 0-carotene, asthaxanthin or a combination thereof. In other embodiments, the colorant is an ingredient extracted from a natural source. The colorant can be charcoal black, annatto, caramel, carmine, chlorophyllin Cu complex, guaiazulene, henna, guanine, spirulina, chlorophyte (green seaweed), blue pea, or a combination thereof.

β-Carotene is an organic, strongly coloured red-orange pigment abundant in fungi, plants, and fruits. It is a member of the carotenes, which are terpenoids, synthesized biochemically from eight isoprene units and thus having 40 carbons. Among the carotenes, 0-carotene is distinguished by having beta-rings at both ends of the molecule.

Astaxanthin (3,3′-dihydroxy-β,β-carotene-4,4′-dione) is a common nutritional, organic red pigment. ATX possess the empiric formula of (C₄₀H₅₂O₄) and is produced by microorganisms such as fungi and algae, and is also found in marine animals (e.g. salmon, crustaceans) providing them with their distinct reddish colour. It is a keto-carotenoid. It belongs to a larger class of chemical compounds known as terpenes built from five carbon precursors, isopentenyl diphosphate, and dimethylallyl diphosphate. Astaxanthin is classified as a xanthophyll, carotenoid compounds that have oxygen-containing components, hydroxyl or ketone, such as zeaxanthin and canthaxanthin.

Advantageously, the colorant provides a visual cue to a user that a surface is coated with an anti-microbial coating. As the active component in the formulation is also able to degrade dyes, the intensity of the coloration will decrease as the anti-microbial effect decreases. This provides confidence to the user and also provides an indication for reapplication of the coating.

Further advantageously, β-carotene and astaxanthin can have potent bioactivity. For example, β-carotene can have significant antibacterial activity against P. syringae, P. carotovorum, and B. subtilis. For example, ATX can significantly inhibit the growth of both Gram-positive and -negative pathogens.

In some embodiments, a weight ratio of the colorant is about 0.01 wt % to about 10 wt % relative to the formulation. In other embodiments, the weight ratio is about 0.01 wt % to about 9 wt %, about 0.01 wt % to about 8 wt %, about 0.01 wt % to about 7 wt %, about 0.01 wt % to about 6 wt %, about 0.05 wt % to about 10 wt %, about 0.1 wt % to about 10 wt %, about 0.1 wt % to about 9 wt %, about 0.1 wt % to about 8 wt %, about 0.1 wt % to about 7 wt %, about 0.1 wt % to about 6 wt %, or about 0.1 wt % to about 5 wt %.

In some embodiments, the natural ingredient formulation further comprises a humectant. A humectant is a hygroscopic substance used to keep things moist. It is commonly used in moisturizer or emollient for protecting, moisturizing, and lubricating the skin. These functions are normally performed by sebum produced by healthy skin.

In some embodiments, the humectant is selected from glycerine, urea, pyrrolidine carboxylic acid, aloe vera or a combination thereof. In other embodiments, the humectant is glycerine.

In some embodiments, a weight ratio of the humectant to the carbohydrate is about 50:15 to about 150:15. In other embodiments, the weight ratio is about 60:15 to about 150:15, about 70:15 to about 150:15, about 80:15 to about 150:15, about 90:15 to about 150:15, about 100:15 to about 150:15, about 60:15 to about 100:15, about 60:15 to about 90:15, about 70:15 to about 90:15, or about 70:15 to about 80:15.

In some embodiments, a weight ratio of the humectant is about 2 wt % to about 60 wt % relative to the formulation. In other embodiments, the weight ratio is about 2 wt % to about 55 wt %, about 2 wt % to about 50 wt %, about 2 wt % to about 45 wt %, about 2 wt % to about 40 wt %, about 2 wt % to about 35 wt %, about 2 wt % to about 30 wt %, about 2 wt % to about 25 wt %, about 2 wt % to about 20 wt %, about 2 wt % to about 18 wt %, about 2 wt % to about 16 wt %, about 2 wt % to about 14 wt %, about 2 wt % to about 12 wt %, about 2 wt % to about 10 wt %, about 2 wt % to about 9 wt %, about 2 wt % to about 8 wt %, about 2 wt % to about 7 wt %, about 2 wt % to about 6 wt %, or about 2 wt % to about 5 wt %.

In some embodiments, the natural ingredient formulation further comprises a fragrance. The fragrance or perfume can be a mixture of fragrant essential oils or aroma compounds. The fragrance can be provided in the form of an essential oil or a mixture thereof. For example, the essential oil can be selected from lavender, peach, Agar oil or oodh, Ajwain oil, Angelica root oil, Anise oil, licorice, Asafoetida oil, Balsam, Basil oil, Bay oil, Bergamot oil, Black pepper oil, Buchu oil, Birch oil, Camphor oil, Cannabis flower essential oil, Calamodin oil or calamansi essential oil, Caraway seed oil, Cardamom seed oil, Carrot seed oil, Cedar oil (or cedarwood oil), Chamomile oil, Calamus oil, Cinnamon oil, Cistus ladanifer, Citron oil, Citronella oil, Clary Sage oil, Coconut oil, Clove oil Coffee oil, Coriander oil, Costmary oil (bible leaf oil), Costus root oil, Cranberry seed oil, Cubeb oil, Cumin seed oil/black seed oil, Cypress oil, Cypriol oil, Curry leaf oil, Davana oil, Dill oil, Elecampane oil, Elemi oil, Eucalyptus oil, Fennel seed oil, Fenugreek oil, Fir oil, Frankincense oil, Galangal oil, Galbanum oil, Garlic oil, Geranium oil, Ginger oil, Goldenrod oil, Grapefruit oil, Henna oil, Helichrysum oil, Hickory nut oil, Horseradish oil, Hyssop, Idaho-grown Tansy, Jasmine oil, Juniper berry oil, Laurus nobilis, Lavender oil, Ledum, Lemon oil, Lemongrass, Lime, Litsea cubeba oil, Linalool, Mandarin, Marjoram, Melissa oil (Lemon balm), Mentha arvensis oil, mint oil, Moringa oil, Mountain Savory, Mugwort oil, Mustard oil, Myrrh oil, Myrtle, Neem oil or neem tree oil, Neroli (bitter orange), Nutmeg oil, Orange oil, Oregano oil, Orris oil, Palo Santo, Parsley oil, Patchouli oil, Perilla essential oil, Pennyroyal oil, Peppermint oil, Petitgrain, Pine oil, Ravensara Red Cedar, Roman Chamomile, Rose oil, Rosehip oil, Rosemary oil, Rosewood oil, Sage oil, star anise oil, Sandalwood oil, Sassafras oil, Savory oil, Schisandra oil, Spearmint oil, Spikenard, Spruce oil, Tangerine, Tarragon oil, Tea tree oil, Thyme oil, Tsuga, Turmeric, Valerian, Warionia, Vetiver oil (khus oil), Western red cedar, Wintergreen, Yarrow oil, Ylang-ylang, yuzu oil, yuzu seed oil, mandarin peel oil or a combination thereof.

In some embodiments, the fragrance is provided in an encapsulated form. In this regard, the fragrance can be enclosed within a micro-sized particle, inside a micrometric shell made up of hard or soft polymer film. The shell can be broken when the formulation is applied to a surface, to release the fragrance. Alternatively, the shell can have a porous structure that can release the fragrance in a controlled manner. Examples of polymers for forming the shell are, but is not limited to, ethyl cellulose, polyvinyl alcohol, gelatin, sodium alginate, or a combination thereof. This advantageously provides for a formulation which is both long lasting in anti-microbial activity as well as in olfactory reception.

In some embodiments, a weight ratio of the fragrance to the carbohydrate is about 1:15 to about 60:15. In other embodiments, the weight ratio is about 1:15 to about 55:15, about 1:15 to about 50:15, about 1:15 to about 45:15, about 1:15 to about 40:15, about 1:15 to about 35:15, about 1:15 to about 30:15, about 1:15 to about 25:15, about 1:15 to about 20:15, about 1:15 to about 18:15, 1:15 to about 16:15, 1:15 to about 14:15, 1:15 to about 12:15, 1:15 to about 10:15, 1:15 to about 8:15, 1:15 to about 6:15, 1:15 to about 4:15, or 1:15 to about 2:15.

In some embodiments, a weight ratio of the fragrance is about 0.01 wt % to about 40 wt % relative to the formulation. In other embodiments, the weight ratio is about 0.01 wt % to about 35 wt %, about 0.01 wt % to about 30 wt %, about 0.01 wt % to about 25 wt %, about 0.01 wt % to about 20 wt %, about 0.01 wt % to about 15 wt %, about 0.01 wt % to about 10 wt %, about 0.01 wt % to about 5 wt %, about 0.01 wt % to about 4 wt %, about 0.01 wt % to about 3 wt %, about 0.01 wt % to about 2 wt %, about 0.01 wt % to about 1 wt %, about 0.01 wt % to about 0.5 wt %, or about 0.01 wt % to about 0.1 wt %.

In some embodiments, the natural ingredient formulation further comprises a permeabilizer. The permeabilizer acts to make the formulation permeable or more permeable to the microbial. The permeabilizer can be selected from polyethylenimine (PEI) and lactic acid.

Polyethylenimine (PEI) is a polymer with repeating unit composed of the amine group and two carbon aliphatic CH₂CH₂ spacer. Linear PEIs contain all secondary amines, in contrast to branched PEIs which contain primary, secondary and tertiary amino groups. Totally branched, dendrimeric forms can also be used and are included within this scope. Co-polymers and block co-polymers comprising PEI are also included within this scope.

For example, poly(ethylene glycol)-block-polyethyleneimine can be used.

In some embodiments, the PEI has a molecular weight of about 1000 Da to about 50000 Da. The molecular weight can be measured by, for example, gel permeation chromatography (GPC). In other embodiments, the molecular weight is about 1000 Da to about 50000 Da, about 1000 Da to about 45000 Da, about 1000 Da to about 40000 Da, about 1000 Da to about 35000 Da, about 1000 Da to about 30000 Da, about 1000 Da to about 25000 Da, about 1000 Da to about 20000 Da, about 1000 Da to about 15000 Da, about 1000 Da to about 10000 Da, about 2000 Da to about 10000 Da, about 3000 Da to about 10000 Da, about 4000 Da to about 10000 Da, about 5000 Da to about 10000 Da, about 6000 Da to about 10000 Da, about 7000 Da to about 10000 Da, or about 8000 Da to about 10000 Da.

In some embodiments, a weight ratio of the permeabilizer to the carbohydrate is about 0.1:15 to about 40:15. In other embodiments, the weight ratio is about 0.1:15 to about 35:15, about 0.1:15 to about 30:15, about 0.1:15 to about 25:15, about 0.1:15 to about 20:15, about 0.1:15 to about 15:15, about 0.1:15 to about 10:15, about 0.1:15 to about 5:15, about 0.1:15 to about 4:15, about 0.1:15 to about 3:15, about 0.1:15 to about 2:15, or about 0.5:15 to about 2:15.

In some embodiments, a weight ratio of the permeabilizer is about 0.01 wt % to about 25 wt % relative to the formulation. In other embodiments, the weight ratio is about 0.01 wt % to about 20 wt %, about 0.01 wt % to about 15 wt %, about 0.01 wt % to about 10 wt %, about 0.01 wt % to about 8 wt %, about 0.01 wt % to about 5 wt %, about 0.01 wt % to about 4 wt %, about 0.01 wt % to about 3 wt %, about 0.01 wt % to about 2 wt %, about 0.01 wt % to about 1 wt %, about 0.01 wt % to about 0.5 wt %, or about 0.01 wt % to about 0.1 wt %.

In some embodiments, the natural ingredient formulation further comprises a surfactant. Surfactants are molecules that spontaneously bond with each other to form vesicles. Surfactants are compounds that lower the surface tension (or interfacial tension) between two liquids, between a gas and a liquid, or between a liquid and a solid. Surfactants may act as detergents, wetting agents, emulsifiers, foaming agents, or dispersants, and in general has a hydrophilic head and a hydrophobic tail. The surfactant can be anionic, cationic, amphoteric or non-Ionic. The surfactant can be selected from a cocoamphoacetate salt, sodium lauryl sulfate, ammonium lauryl sulfate, sodium laurethsulfate, taurates, isethionates, olefin sulfonates, sulfosuccinates, cetrimonium chloride, stearalkonium chloride, sodium lauriminodipropionate, disodium lauroamphodiacetate, cetyl or stearyl alcohol, polysorbate esters and the likes. For example, disodium cocoamphodiacetate (DSCADA) is a synthetic amphoteric surfactant. In some embodiments, the surfactant is decyl glucoside.

In some embodiments, a weight ratio of the surfactant to the carbohydrate is about 5:15 to about 750:15. In other embodiments, the weight ratio is about 5:15 to about 700:15, about 5:15 to about 650:15, about 5:15 to about 600:15, about 5:15 to about 550:15, about 5:15 to about 500:15, about 5:15 to about 450:15, about 5:15 to about 400:15, about 5:15 to about 350:15, about 5:15 to about 300:15, about 5:15 to about 250:15, about 5:15 to about 200:15, about 5:15 to about 150:15, about 5:15 to about 100:15, about 5:15 to about 50:15, about 5:15 to about 20:15, about 5:15 to about 18:15, about 5:15 to about 16:15, about 5:15 to about 14:15, about 5:15 to about 12:15, about 7:15 to about 12:15, or about 8:15 to about 12:15.

In some embodiments, a weight ratio of the surfactant is about 0.5 wt % to about 80 wt % relative to the formulation. In other embodiments, the weight ratio is about 0.5 wt % to about 70 wt %, about 0.5 wt % to about 60 wt %, about 0.5 wt % to about 50 wt %, about 0.5 wt % to about 40 wt %, about 0.5 wt % to about 30 wt %, about 0.5 wt % to about 20 wt %, about 0.5 wt % to about 10 wt %, about 0.5 wt % to about 5 wt %, about 0.5 wt % to about 2 wt %, about 0.5 wt % to about 1.5 wt %, about 0.5 wt % to about 1 wt %, about 0.5 wt % to about 0.9 wt %, or about 0.5 wt % to about 0.8 wt %.

In some embodiments, the natural ingredient formulation further comprises a film forming agent. Film-forming agents are substances that leave a cohesive, and continuous covering over a surface when applied to the surface. The film can have strong hydrophilic properties such that it gives a smooth feeling touched. Examples of film-forming agents include polyvinylpyrrolidone (PVP), acrylates, acrylamides, and copolymers. In some embodiments, the formulation further comprises a film forming agent selected from (3-glycidyloxypropyl)trimethoxysilane and/or gelatin.

In some embodiments, a weight ratio of the film forming agent is about 0.01 wt % to about 10 wt % relative to the formulation. In other embodiments, the weight ratio is about 0.01 wt % to about 9 wt %, about 0.01 wt % to about 8 wt %, about 0.01 wt % to about 7 wt %, about 0.01 wt % to about 6 wt %, about 0.01 wt % to about 5 wt %, about 0.01 wt % to about 4 wt %, about 0.01 wt % to about 3 wt %, about 0.05 wt % to about 3 wt %, about 0.1 wt % to about 3 wt %, about 0.1 wt % to about 2 wt %, about 0.1 wt % to about 1 wt %, or about 0.5 wt % to about 1 wt %. For example, (3-glycidyloxypropyl)trimethoxysilane and/or gelatin can be added at about 0.5 wt % to about 1 wt %.

In some embodiments, a weight ratio of the film forming agent is about 10 wt % to about 25 wt % relative to the formulation. In other embodiments, the weight ratio is about 12 wt % to about 25 wt %, about 14 wt % to about 25 wt %, about 16 wt % to about 25 wt %, about 18 wt % to about 25 wt %, or about 18 wt % to about 20 wt %.

Advantageously, it was found that addition of a film forming agent enhances the ability of the anti-microbial effect. The film forming agent also allows for improved coating on metal surfaces. This allows the formulation to be applied as a long term coating for up to 6 months.

In some embodiments, the natural ingredient formulation further comprises a solvent. The solvent can be an aqueous medium. The solvent can be for example water, and/or ethyl acetate.

The term “aqueous solution” or “aqueous medium” used herein refers to a water based solvent or solvent system, and which comprises of mainly water. Such solvents can be either polar or non-polar, and/or either protic or aprotic. Solvent systems refer to combinations of solvents which resulting in a final single phase. Both ‘solvents’ and ‘solvent systems’ can include, and is not limited to, pentane, cyclopentane, hexane, cyclohexane, benzene, toluene, dioxane, chloroform, diethylether, dichloromethane, tetrahydrofuran, ethyl acetate, acetone, dimethylformamide, acetonitrile, dimethyl sulfoxide, nitromethane, propylene carbonate, formic acid, butanol, isopropanol, propanol, ethanol, methanol, acetic acid, ethylene glycol, diethylene glycol or water. Water based solvent or solvent systems can also include dissolved ions, salts and molecules such as amino acids, proteins, sugars and phospholipids. Such salts may be, but not limited to, sodium chloride, potassium chloride, ammonium acetate, magnesium acetate, magnesium chloride, magnesium sulfate, potassium acetate, potassium chloride, sodium acetate, sodium citrate, zinc chloride, HEPES sodium, calcium chloride, ferric nitrate, sodium bicarbonate, potassium phosphate and sodium phosphate. As such, biological fluids, physiological solutions and culture medium also fall within this definition. In most embodiments, the aqueous solution is water. In some embodiments, the aqueous solution is deionised water. In some embodiments, the aqueous solution is Millipore water.

In some embodiments, a weight ratio of the solvent to the carbohydrate is about 100:15 to about 3000:15. In other embodiments, the weight ratio is about 100:15 to about 2500:15, about 100:15 to about 2000:15, about 100:15 to about 1500:15, about 200:15 to about 1500:15, about 300:15 to about 1500:15, about 400:15 to about 1500:15, about 500:15 to about 1500:15, about 600:15 to about 1500:15, about 700:15 to about 1500:15, or about 700:15 to about 1200:15.

In some embodiments, the natural ingredient formulation further comprises an anti-oxidant. The anti-oxidant can, for example, be oxalic acid, phytic acid, tannins, ascorbic acid, glutathione, lipoic acid, uric acid, carotenes, ubiquinol, and α-tocopherol. Another example of an anti-oxidant is butylated hydroxytoluene.

Advantageously, it was found that the addition of an anti-oxidant can help “preserve” or at least slow down the free radical generation of the iron particles. This improves the shelf-life of the formulation while at the same time does not cause a decrease in its anti-microbial efficacy.

In some embodiments, a weight ratio of the anti-oxidant is about 0.01 wt % to about 5 wt % relative to the formulation. In other embodiments, the weight ratio is about 0.01 wt % to about 4 wt %, about 0.01 wt % to about 3 wt %, about 0.01 wt % to about 2 wt %, about 0.01 wt % to about 1 wt %, about 0.01 wt % to about 0.5 wt %, or about 0.01 wt % to about 0.1 wt %.

In some embodiments, the formulation further comprises cellulose. The cellulose can be a powdered cellulose, having a particle size of about 1 μm to about 100 μm. The cellulose can be extracted from a fruit rind and/or a symbiotic culture of bacteria and yeast (SCOBY). SCOBY is an ingredient used in the fermentation and production of kombucha. In some embodiments, the cellulose is extracted from durian rind.

The addition of powdered cellulose produces a sustainable refill powder which can be easily transported. The use of cellulose from food waste can reduce the carbon footprint due to reduced energy needed for transportation. With using recyclable bottles and powder refills packs, more than 1 millions plastic bottles could be reduced yearly, making the switch to refillable cleaning. The user can thus simply add water to the refill powder to reconstitute the formulation as a liquid for use.

Additionally, it was found that cellulose extracted from durian rind and/or SCOBY has an antimicrobial activity 0.3 log reduction compared to cellulose derived from other sources. The addition of cellulose extracted from durian rind and/or SCOBY can synergistically (or at least additively) improve the antimicrobial activity of the formulation.

In some embodiments, a weight ratio of the cellulose is about 1 wt % to about 20 wt % relative to the formulation. In other embodiments, the weight ratio is about 1 wt % to about 15 wt %, about 2 wt % to about 15 wt %, about 4 wt % to about 15 wt %, about 6 wt % to about 15 wt %, about 8 wt % to about 15 wt %, about 10 wt % to about 15 wt %, or about 12 wt % to about 15 wt %.

The formulation can further comprise maltodextrin. Maltodextrin can improve the flowability of the refill powder.

In some embodiments, a weight ratio of maltodextrin is about 1 wt % to about 20 wt % relative to the formulation. In other embodiments, the weight ratio is about 1 wt % to about 15 wt %, about 2 wt % to about 15 wt %, about 4 wt % to about 15 wt %, about 6 wt % to about 15 wt %, about 8 wt % to about 15 wt %, about 10 wt % to about 15 wt %, or about 12 wt % to about 15 wt %.

Natural Ingredient Formulation

In some embodiments, the natural ingredient formulation has a pH of about 4 to about 5. The pH can be regulated through a controlled addition of acetic acid or any other pH buffers. It was found that when the pH is in this range, the radicals are further advantageously more stable.

In some embodiments, the natural ingredient formulation has an at least about 2 log reduction against E. coli after 5 min. In other embodiments, the natural ingredient formulation has an at least about 3 log reduction, about 4 log reduction, or about 5 log reduction.

In some embodiments, the natural ingredient formulation has an at least about 2 log reduction against E. coli after 1 min. In other embodiments, the natural ingredient formulation has an at least about 3 log reduction, about 4 log reduction, or about 5 log reduction.

In some embodiments, the natural ingredient formulation has an at least about 2 log reduction against S. Aureus after 5 min. In other embodiments, the natural ingredient formulation has an at least about 3 log reduction, about 4 log reduction, or about 5 log reduction.

In some embodiments, the natural ingredient formulation has an at least about 2 log reduction against S. Aureus after 1 min. In other embodiments, the natural ingredient formulation has an at least about 3 log reduction, about 4 log reduction, or about 5 log reduction.

In some embodiments, the formulation has an antiviral activity rate of at least about 90%. In other embodiments, the antiviral activity rate is at least about 91%, about 92%, about 93%, about 94%, about 95%, about 96%, about 97%, about 98%, or about 99%. The formulation can have an antiviral activity against viruses such as HCoV-229E Virus, murine-hepatitis virus and/or H3N2 virus.

The present invention provides a method of using the formulation in anti-microbial applications. The formulation as disclosed herein can be in any applicable form. For example, the formulation can be made into a gel, liquid, or be in a sprayable form.

In some embodiments, the natural ingredient formulation is for use as an antimicrobial coating, disinfectant, hand sanitizer, and/or soap. The formulation can also be used in detergents, aerosols, all-purpose cleaners, pest control solutions, and dish washing liquid.

For example, to be suitable for use as a spray, a viscosity reducing agent can be added in order to have a final product with a viscosity of about 80,000 cPs to about 900,000 cPs. As the iron particles are of a nano and/or micron size, they are able to be dispersed and suspended in air for a suitable duration. When sprayed on a surface, the non-aggregated iron particles can also homogenously dispersed on the surface.

The formulation can be used in air purification as its able to decompose harmful particle matter, volatile organic compounds, polyaromatic hydrocarbons when these entities come in contact with, for example, a treated surfaces. The formulation can also be used to form a coating for air filters and filtration systems as an additional layer of security.

The formulation can also be used in combination with a resin or polymer such as varnish to form an anti-microbial coating. The formulation can also be used in combination with a resin or polymer such as varnish to form a stain prevention coating.

The formulation can also be used in waste water treatment or management. For example, the composition can be added to waste water in order to kill the microbes and/or reduce aromatic colorants and impurities.

It can also be used for water purification. In some embodiments, the formulation can provide at least a 60% decrease in dye coloration. In some embodiments, the formulation can provide at least a 60% decrease in Brilliant Blue R coloration.

In some embodiments, the formulation is applied on at least a surface of a fabric for use as a wet wipe. The formulation can be homogenously applied to the fabric by immersing the fabric in a solution of the formulation. The fabric can be impregnated by the formulation.

In some embodiments, a weight ratio of the formulation to the fabric is about 2:1 to about 10:1. In other embodiments, the weight ratio is about 2:1 to about 9:1, about 2:1 to about 8:1, about 2:1 to about 7:1, about 2:1 to about 6:1, or about 2:1 to about 5:1.

In some embodiments, the fabric is a porous fabric. In other embodiments, the fabric is a nonwoven fabric. Nonwoven fabric is a fabric-like material made from staple fibre (short) and long fibres (continuous long), bonded together by chemical, mechanical, heat or solvent treatment. Examples of nonwoven fabric includes polyester or polypropylene.

In some embodiments, the fabric comprises cellulose extracted from a fruit rind and/or a symbiotic culture of bacteria and yeast (SCOBY). SCOBY is an ingredient used in the fermentation and production of kombucha. In some embodiments, the cellulose is extracted from durian rind. In some embodiments, the fabric further comprises bamboo fiber.

There is a tremendous amount of durian rind waste produced during the durian season. Singaporeans consumed six million durians in the first six months of 2018 alone, which is equivalent to approximately twelve million durians consumed in a year. The durian rind makes up 60% of the entire durian fruit. This is discarded as waste and incinerated and could cause environmental problems if not disposed of properly. Approximately, 14,000 tons of durian husks are incinerated each year. It has been studied that the durian rind contains 31-35% cellulose. This means approximately one third (30%) of the durian rind could be transformed into cellulose. Another food waste rich in cellulose is SCOBY, a by-product from kombucha tea production. SCOBY contains approximately 90% cellulose.

As mentioned above, cellulose extracted from durian rind and/or SCOBY shows an antimicrobial activity of about 0.3 log reduction. Accordingly, the present invention also provides a fabric comprising cellulose extracted from a fruit rind and/or a symbiotic culture of bacteria and yeast (SCOBY).

The present invention also provides a method of extracting cellulose from fruit rind and/or SCOBY. For example, the method can comprise a freeze drying step, a grinding and/or milling step, a cellulose extraction step, and a drying step. The cellulose can be extracted by dispersing the milled sample in a solution and centrifuging to separate the cellulose. Alternatively, a bioreactor can be used.

The present invention also provides a method of disinfecting a surface, comprising use of a natural ingredient formulation as disclosed herein. For example, the natural ingredient formulation can be sprayed onto the surface.

As used herein, ‘disinfecting’ refers to an action of cleaning something in order to destroy microorganisms, such as bacteria and/or viruses.

The present invention also provides a method of coating a surface with an anti-microbial coating, comprising use of a natural ingredient formulation as disclosed herein. The anti-microbial coating can be for use as a long term coating, for example, more than 1 months. For example, the natural ingredient formulation can be spray coated or painted onto the surface.

In some embodiments, when used as a surface disinfectant or coating, the natural ingredient formulation comprises:
a) cashew testa extract;
b) an iron particle and/or iron oxide particle;
c) a carbohydrate; and
d) a permeabilizer;
wherein a weight ratio of the cashew testa extract to the iron particle and/or iron oxide particle is about 100:1 to about 1:100;
wherein a weight ratio of the carbohydrate (c) to the cashew testa extract and iron particle and/or iron oxide particle (a and b) is about 15:1 to about 15:14;
wherein the cashew testa extract at least partially incorporated in the iron particle and/or iron oxide particle; and
wherein a weight ratio of the permeabilizer is 0.01 wt % to about 5 wt % relative to the formulation.

In some embodiments, when used as a surface disinfectant or coating, the natural ingredient formulation comprises:
a) cashew testa extract;
b) an iron particle and/or iron oxide particle;
c) a carbohydrate; and
d) a permeabilizer;
wherein a weight ratio of the cashew testa extract to the iron particle and/or iron oxide particle is about 100:1 to about 1:100;
wherein a weight ratio of the carbohydrate (c) to the cashew testa extract and iron particle and/or iron oxide particle (a and b) is about 8:1 to about 1:1;
wherein the cashew testa extract at least partially incorporated in the iron particle and/or iron oxide particle; and
wherein a weight ratio of the permeabilizer is 0.01 wt % to about 25 wt % relative to the formulation.

In some embodiments, when used as a surface disinfectant or coating, the natural ingredient formulation comprises:
a) cashew testa extract;
b) an iron particle and/or iron oxide particle;
c) a carbohydrate; and
d) a permeabilizer;
wherein a weight ratio of the cashew testa extract to the iron particle and/or iron oxide particle is about 100:1 to about 1:200;
wherein a weight ratio of the carbohydrate (c) to the cashew testa extract and iron particle and/or iron oxide particle (a and b) is about 8:1 to about 1:300;
wherein the cashew testa extract at least partially incorporated in the iron particle and/or iron oxide particle; and
wherein a weight ratio of the permeabilizer is 0.01 wt % to about 25 wt % relative to the formulation.

In some embodiments, when used as a surface disinfectant or coating, the natural ingredient formulation comprises:
a) cashew testa extract;
b) an iron particle and/or iron oxide particle;
c) chitosan; and
d) PEI;
wherein a weight ratio of the cashew testa extract to the iron particle and/or iron oxide particle is about 100:1 to about 1:200;
wherein a weight ratio of chitosan to the cashew testa extract and iron particle and/or iron oxide particle is about 8:1 to about 1:300;
wherein the cashew testa extract at least partially incorporated in the iron particle and/or iron oxide particle; and
wherein a weight ratio of PEI is about 0.01 wt % to about 25 wt % relative to the formulation.

In some embodiments, when used as a surface disinfectant or coating, the natural ingredient formulation comprises:
a) cashew testa extract;
b) an iron particle and/or iron oxide particle;
c) a carbohydrate;
d) a permeabilizer; and
e) a film forming agent;
wherein a weight ratio of the cashew testa extract to the iron particle and/or iron oxide particle is about 100:1 to about 1:200;
wherein a weight ratio of the carbohydrate to the cashew testa extract and iron particle and/or iron oxide particle is about 8:1 to about 1:300;
wherein the cashew testa extract at least partially incorporated in the iron particle and/or iron oxide particle;
wherein a weight ratio of the permeabilizer is about 0.01 wt % to about 25 wt % relative to the formulation; and
wherein a weight ratio of the film forming agent is about 10 wt % to about 25 wt % relative to the formulation.

In some embodiments, when used as a surface disinfectant or coating, the natural ingredient formulation comprises:
a) cashew testa extract;
b) an iron particle and/or iron oxide particle;
c) chitosan;
d) PEI; and
e) gelatin;
wherein a weight ratio of the cashew testa extract to the iron particle and/or iron oxide particle is about 100:1 to about 1:200;
wherein a weight ratio of chitosan to the cashew testa extract and iron particle and/or iron oxide particle is about 30:1 to about 1:300;
wherein the cashew testa extract is at least partially incorporated in the iron particle and/or iron oxide particle;
wherein a weight ratio of PEI is about 0.01 wt % to about 5 wt % relative to the formulation; and
wherein a weight ratio of gelatin is about 0.01 wt % to about 10 wt % relative to the formulation.

In some embodiments, when used as a surface disinfectant or coating, the natural ingredient formulation comprises:
a) cashew testa extract;
b) an iron particle and/or iron oxide particle;
c) chitosan;
d) PEI;
e) gelatin; and
f) 3-glycidoxypropyltrimethoxysilane;
wherein a weight ratio of the cashew testa extract to the iron particle and/or iron oxide particle is about 100:1 to about 1:200;
wherein a weight ratio of chitosan to the cashew testa extract and iron particle and/or iron oxide particle is about 30:1 to about 1:300;
wherein the cashew testa extract is at least partially incorporated in the iron particle and/or iron oxide particle;
wherein a weight ratio of PEI is about 0.01 wt % to about 5 wt % relative to the formulation; and
wherein a combined weight ratio of gelatin and 3-glycidoxypropyltrimethoxysilane is about 0.01 wt % to about 10 wt % relative to the formulation.

In some embodiments, when used as a surface disinfectant or coating, the natural ingredient formulation comprises:
a) cashew testa extract;
b) an iron particle and/or iron oxide particle;
c) chitosan;
d) PEI; and
e) 3-glycidoxypropyltrimethoxysilane;
wherein a weight ratio of the cashew testa extract to the iron particle and/or iron oxide particle is about 100:1 to about 1:200;
wherein a weight ratio of chitosan to the cashew testa extract and iron particle and/or iron oxide particle is about 8:1 to about 1:300;
wherein the cashew testa extract is at least partially incorporated in the iron particle and/or iron oxide particle;
wherein a weight ratio of PEI is about 0.01 wt % to about 25 wt % relative to the formulation; and
wherein a weight ratio of 3-glycidoxypropyltrimethoxysilane is about 10 wt % to about 25 wt % relative to the formulation.

The present invention also provides a method of sanitizing a user's hand, comprising use of a natural ingredient formulation as disclosed herein. For example, the natural ingredient formulation can be provided in the form of a hand rub or ethanol/water spray.

In some embodiments, when used as a hand sanitizer, the natural ingredient formulation comprises:
a) cashew testa extract;
b) an iron particle and/or iron oxide particle;
c) a carbohydrate; and
d) a humectant;
wherein a weight ratio of the cashew testa extract to the iron particle and/or iron oxide particle is about 100:1 to about 1:200;
wherein a weight ratio of the carbohydrate to the cashew testa extract and iron particle and/or iron oxide particle is about 30:1 to about 1:300;
wherein the cashew testa extract is at least partially incorporated in the iron particle and/or iron oxide particle; and
wherein a weight ratio of the humectant is about 2 wt % to about 60 wt % relative to the formulation.

In some embodiments, when used as a hand sanitizer, the natural ingredient formulation comprises:
a) cashew testa extract;
b) an iron particle and/or iron oxide particle;
c) a carbohydrate; and
d) a humectant;
wherein a weight ratio of the cashew testa extract to the iron particle and/or iron oxide particle is about 100:1 to about 1:100;
wherein a weight ratio of the carbohydrate to the cashew testa extract and iron particle and/or iron oxide particle is about 15:1 to about 15:14;
wherein the cashew testa extract is at least partially incorporated in the iron particle and/or iron oxide particle; and
wherein a weight ratio of the humectant is about 2 wt % to about 60 wt % relative to the formulation.

In some embodiments, when used as a hand sanitizer, the natural ingredient formulation comprises:
a) cashew testa extract;
b) an iron particle and/or iron oxide particle;
c) chitosan; and
d) glycerine;
wherein a weight ratio of the cashew testa extract to the iron particle and/or iron oxide particle is about 100:1 to about 1:200;
wherein a weight ratio of chitosan to the cashew testa extract and iron particle and/or iron oxide particle is about 30:1 to about 1:300;
wherein the cashew testa extract is at least partially incorporated in the iron particle and/or iron oxide particle; and
wherein a weight ratio of the glycerine is about 2 wt % to about 60 wt % relative to the formulation.

In some embodiments, when used as a hand sanitizer, the natural ingredient formulation comprises:
a) cashew testa extract;
b) an iron particle and/or iron oxide particle;
c) a carbohydrate;
d) a humectant; and
e) a fragrance;
wherein a weight ratio of the cashew testa extract to the iron particle and/or iron oxide particle is about 100:1 to about 1:200;
wherein a weight ratio of the carbohydrate to the cashew testa extract and iron particle and/or iron oxide particle is about 30:1 to about 1:300;
wherein the cashew testa extract is at least partially incorporated in the iron particle and/or iron oxide particle; and
wherein a weight ratio of the humectant is about 2 wt % to about 60 wt % relative to the formulation.

In some embodiments, when used as a hand sanitizer, the natural ingredient formulation comprises:
a) cashew testa extract;
b) an iron particle and/or iron oxide particle;
c) chitosan;
d) glycerine; and
e) a fragrance;
wherein a weight ratio of the cashew testa extract to the iron particle and/or iron oxide particle is about 100:1 to about 1:200;
wherein a weight ratio of chitosan to the cashew testa extract and iron particle and/or iron oxide particle is about 30:1 to about 1:300;
wherein the cashew testa extract is at least partially incorporated in the iron particle and/or iron oxide particle; and
wherein a weight ratio of the glycerine is about 2 wt % to about 60 wt % relative to the formulation.

In some embodiments, when used as a hand sanitizer, the natural ingredient formulation comprises:
a) cashew testa extract;
b) an iron particle and/or iron oxide particle;
c) chitosan;
d) glycerine;
e) a fragrance; and
f) a surfactant;
wherein a weight ratio of the cashew testa extract to the iron particle and/or iron oxide particle is about 100:1 to about 1:200;
wherein a weight ratio of chitosan to the cashew testa extract and iron particle and/or iron oxide particle is about 30:1 to about 1:300;
wherein the cashew testa extract is at least partially incorporated in the iron particle and/or iron oxide particle;
wherein a weight ratio of the glycerine is about 2 wt % to about 60 wt % relative to the formulation; and a weight ratio of the surfactant is about 0.5 wt % to about 20 wt % relative to the formulation.

The present invention also provides a method of providing an anti-microbial function to a textile, comprising use of a natural ingredient formulation as disclosed herein. For example, the natural ingredient formulation can be provided as a soap for washing the textile, the process of which imparts the natural ingredient formulation and hence anti-microbial function to the textile.

In some embodiments, the natural ingredient formulation, comprises:
a) cashew testa extract;
b) an iron particle and/or iron oxide particle;
c) a carbohydrate; and
d) a surfactant;
wherein a weight ratio of the cashew testa extract to the iron particle and/or iron oxide particle is about 100:1 to about 1:200;
wherein a weight ratio of the carbohydrate to the cashew testa extract and iron particle and/or iron oxide particle is about 30:1 to about 1:300;
wherein the cashew testa extract is at least partially incorporated in the iron particle and/or iron oxide particle; and
wherein a weight ratio of the surfactant is about 0.5 wt % to about 80 wt % relative to the formulation

In some embodiments, the natural ingredient formulation, comprises:
a) cashew testa extract;
b) an iron particle and/or iron oxide particle;
c) a carbohydrate; and
d) a surfactant;
wherein a weight ratio of the cashew testa extract to the iron particle and/or iron oxide particle is about 100:1 to about 1:100;
wherein a weight ratio of the carbohydrate to the cashew testa extract and iron particle and/or iron oxide particle is about 15:1 to about 15:14;
wherein the cashew testa extract is at least partially incorporated in the iron particle and/or iron oxide particle; and
wherein a weight ratio of the surfactant is about 0.5 wt % to about 80 wt % relative to the formulation.

In some embodiments, the natural ingredient formulation, comprises:
a) cashew testa extract;
b) an iron particle and/or iron oxide particle;
c) chitosan; and
d) a cocoamphoacetate salt;
wherein a weight ratio of the cashew testa extract to the iron particle and/or iron oxide particle is about 100:1 to about 1:200;
wherein a weight ratio of chitosan to the cashew testa extract and iron particle and/or iron oxide particle is about 30:1 to about 1:300;
wherein the cashew testa extract is at least partially incorporated in the iron particle and/or iron oxide particle; and
wherein a weight ratio of the cocoamphoacetate salt is about 0.5 wt % to about 2 wt % relative to the formulation.

The present invention also provides a method of cleaning a non-biological surface, comprising contacting a formulation with the non-biological surface.

As used herein, ‘cleaning’ refers to an action of making something clean by, for example, removing dirt, marks or stains.

As is shown herein, the formulation is also capable of degrading coloured compounds. By means of such degradation, the colour of the compounds is lost due to the disruption of its aromaticity/conjugated system.

In some embodiments, the ROS can be generated in the dark. In other embodiments, the ROS can be generated in the absence of UV radiation. In other embodiments, the ROS generated is selected from ·O₂⁻, H₂O₂, ·OH, ¹O₂, α-O, or a combination thereof.

In some embodiments, when FeCl₃ is used in the synthesis, the presence of Cl⁻ anions can also contribute to the generation of ROS. The ROS can be Cl· and/or Cl₂⁻·. This is further advantageous in that the anti-microbial effect can be enhanced, and in particular, can also extend some distance from an applied surface. In this sense, an anti-microbial effect can be obtained without the microbe being in contact with the surface.

In some embodiments, the ROS are dispersible some distance away from an applied area or surface. In other embodiments, the distance is of about 0.1 mm to about 10 cm. In other embodiments, the distance is about 1 cm, 2 cm, 5 cm, 7 cm, or 10 cm.

Formation of Natural Ingredient Formulation

The present invention also provides a method of forming a natural ingredient formulation, comprising:
a) mixing a cashew testa extract, an iron particle and/or iron oxide particle and a carbohydrate;
wherein a weight ratio of the cashew testa extract to the iron particle and/or iron oxide particle is about 100:1 to about 1:200; and
wherein a weight ratio of the carbohydrate to the cashew testa extract and iron particle and/or iron oxide particle is about 30:1 to about 1:300; and
wherein the cashew testa extract is at least partially incorporated in the iron particle and/or iron oxide particle.

In some embodiments, the method of forming a natural ingredient formulation, comprising:
a) mixing a cashew testa extract, an iron particle and/or iron oxide particle and a carbohydrate;
wherein a weight ratio of the cashew testa extract to the iron particle and/or iron oxide particle is about 100:1 to about 1:100; and
wherein a weight ratio of the carbohydrate to the cashew testa extract and iron particle and/or iron oxide particle is about 15:1 to about 15:14; and
wherein the cashew testa extract is at least partially incorporated in the iron particle and/or iron oxide particle.

In some embodiments, the method further comprises a step after step (a) of adjusting a pH of the natural ingredient formulation to of about 4 to about 5. The pH can be adjusted using acetic acid or any other pH buffers. Examples include, but is not limited to, citric acid, KH₂PO₄, N-Cyclohexyl-2-aminoethanesulfonic acid (CHES), and borates.

In some embodiments, the method further comprises a step after step (a) of adding an excipient selected from colorant, humectant, fragrance, stabilizer, permeabilizer, attachment promoter, transfection agent, surfactant, solvent, anti-oxidant or a combination thereof.

In some embodiments, the method further comprises a step after step (a) of diluting the formulation in an aqueous medium.

In some embodiments, the method further comprises a step after step (a) of filtering the formulation.

As mentioned above, the cashew testa extract and the iron particle and/or iron oxide particle can be physically mixed to form iron particle and/or iron oxide particle passivated with cashew testa extract.

Accordingly, in some embodiments, the mixing of the cashew testa extract with the iron particle and/or iron oxide particle is for the iron oxide particles to be at least partially passivating by the cashew testa extract.

Accordingly, in some embodiments, the method of forming a natural ingredient formulation, comprising:
i) mixing a cashew testa extract and an iron particle and/or iron oxide particle; and
a) mixing the mixture of (i) and a carbohydrate;
wherein a weight ratio of the cashew testa extract to the iron particle and/or iron oxide particle is about 100:1 to about 1:200; and
wherein a weight ratio of the carbohydrate to the cashew testa extract and iron particle and/or iron oxide particle is about 30:1 to about 1:300;
wherein the cashew testa extract is at least partially incorporated in the iron particle and/or iron oxide particle.

Alternatively, the iron particle and/or iron oxide particle can be formed from an iron precursor. For example, the cashew testa extract and an iron precursor can be first chemically reacted to form the iron particle and/or iron oxide particle with cashew testa extract incorporated within. The carbohydrate can be subsequently mixed in.

Accordingly, in some embodiments, the method further comprises a step before step (a) of reacting the cashew testa extract with an iron precursor in order to form the cashew testa extract and the iron particle and/or iron oxide particle.

Accordingly, in some embodiments, the method of forming a natural ingredient formulation, comprising:
i) reacting a cashew testa extract and an iron precursor in order to form the cashew testa extract and an iron particle and/or iron oxide particle; and
a) mixing the cashew testa extract, the iron particle and/or iron oxide particle from step (i) and a carbohydrate;
wherein a weight ratio of the cashew testa extract to the iron particle and/or iron oxide particle is about 100:1 to about 1:200; and
wherein a weight ratio of the carbohydrate to the cashew testa extract and iron particle and/or iron oxide particle is about 30:1 to about 1:300;
wherein the cashew testa extract is at least partially incorporated in the iron particle and/or iron oxide particle.

In other embodiments, the method of forming a natural ingredient formulation, comprising:
i) reacting a cashew testa extract and an iron precursor in order to form the cashew testa extract and an iron particle and/or iron oxide particle;
a) mixing the cashew testa extract, the an iron particle and/or iron oxide particle from step (i) and a carbohydrate;
wherein a weight ratio of the cashew testa extract to the iron oxide nanoparticle is about 100:1 to about 1:200; and
wherein a weight ratio of the carbohydrate to the cashew testa extract and iron oxide nanoparticle is about 30:1 to about 1:300;
wherein the cashew testa extract is at least partially incorporated in the iron particle and/or iron oxide particle.

In some embodiments, the mixing in step (i) or step (a) is performed for at least about 1 h. In other embodiments, the mixing is performed for at least about 2 h, 4 h, 6 h, 12 h, or 24 h.

In some embodiments, the mixing in step (a) is performed at room temperature. In other embodiments, the mixing is performed at about 15° C. to about 30° C., or about 20° C. to about 30° C.

### Examples

Cashew testa extract
Appearance: Brown liquid
Physicochemical characteristics: High radical scavenging activity (antioxidant activity)
Composition: Mixture of compounds including catechin, epicatechin, tannic acid
Thermally stable up 200° C.
Solubility in water is 2850 g/L

### Example 1: Synthesis of Iron-Polyphenol Complex Nanoparticles (Fe-Cashew Core Covered with a Shell of Fe(NO₃)₃·9H₂O) (Composite A)

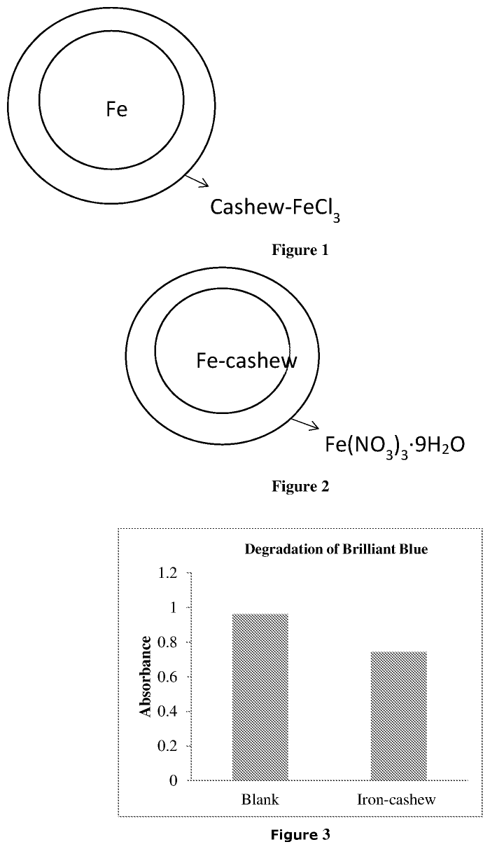

Cashew testa extract and 2 g of iron powder was combined and this mixture was bubbled with nitrogen gas for 1 h. During this process, iron and cashew extract would be linked to form the compound Fe-cashew. Separately, the solution of 0.1 M Fe(NO₃)₃·9H₂O was prepared, and bubbled with nitrogen gas for 1 h. Subsequently, the Fe-cashew solution and 0.5 M Fe(NO₃)₃·9H₂O were combined in a 1:1 volume ratio. The reaction was continued for 24 h under nitrogen, and the product was stored at 4° C. This would produce a Fe-cashew as the core of the nanoparticles, which is covered with a shell of Fe(NO₃)₃·9H₂O. (FIG. 1)

Alternatively, 2 g Fe and 5 ml of cashew extract were combined and incubated at 1 h, room temperature with constant agitation. Then 5 ml of 0.1M FeCl₃ was added and the mixture was incubated at 1 h, room temperature with constant agitation. The entire mixture was centrifuged at 5000 rpm and the black precipitate were collected. It was washed with water and then ethanol.

In the examples as disclosed herein, different types of Fe salts were tested; for example, FeCl₃, FeSO₄, Fe₂(SO₄)₃, Fe(NO₃)₃, Fe(NO₃)₂.

### Example 2: Synthesis of Iron-Polyphenol Complex Nanoparticles (Fe Core Covered with a Shell of Cashew-Fe(NO₃)₃·9H₂O) (Composite B)

2 g of iron powder in water was bubbled with nitrogen gas for 1 h. Separately, 5 ml cashew testa extract and 5 ml 0.1 M Fe(NO₃)₃·9H₂O was combined, and bubbled with nitrogen gas for 1 h to form a cashew-Fe(NO₃)₃·9H₂O linked compound. Subsequently, the iron solution and cashew-Fe(NO₃)₃·9H₂O solution were combined in a 1:1 volume ratio. The reaction was continued for 24 h under nitrogen, and the product was stored at 4° C. This would produce nanoparticle with Fe as the core, which is covered with a shell of cashew-Fe(NO₃)₃·9H₂O. (FIG. 2)

### Example 3: Synthesis of Cashew Extract Activated Iron Powder (Composite C)

Fresh iron powder (4 g) was mixed with cashew testa extract (4 ml), and the mixture was stirred at 80° C. for 24 hours. After cooling to room temperature, solid residuals of the composite C were collected.

### Example 4: Synthesis of Cashew Extract Iron Nanoparticles (Composite D)

A solution of 0.1 M FeCl₃ was prepared by adding 16.23 g of FeCl₃ in 1 litre of Milli-Q water. Subsequently, 0.1 M FeCl₃ solution was added to cashew extract in a 1:1 ratio. The formation of iron-cashew nanoparticles was marked by the appearance of iron black precipitate, and this was collected by centrifuging at 7000 rpm. Then the iron-cashew nanoparticles powders were frozen at −20° C. and then were dried in a freeze-dryer at −45° C. with the pressure at 10 Pa for 24 h.

Alternatively, 0.1 M FeCl₃ solution to cashew extract in a 1:1 ratio can be incubated at 1 h, room temperature. Then the entire mixture was centrifuged at 5000 rpm and the black precipitate were collected. It was washed with water and then ethanol.

### Example 5: Synthesis of Cashew Extract Iron Nanoparticles (Composite E)

0.1 M FeCl₃+cashew testa extract were combined in a 1:1 ratio and incubated at 1 h, room temperature. Then 1 M NaOH was added until the pH was 11. The entire mixture was centrifuged at 5000 rpm and the black precipitate were collected. It was washed with water and then ethanol.

### Example 6: Fe—FeCl₃ as the Core and Cashew as the Shell (Composite F)

2 g Fe and 5 ml to 20 ml of 0.1M to 0.5M FeCl₃ were combined and incubated at 1 h, room temperature with constant agitation. Then 5 ml to 20 ml of cashew extract was added and the mixture was incubated at 1 h, room temperature with constant agitation. The entire mixture was centrifuged at 5000 rpm and the precipitate were collected. It was washed with water and then ethanol.

Energy-Dispersive X-Ray Spectroscopy (EDX) Analysis of Iron Particles and/or Iron Oxide Particles

The EDX results as shown below are based on Example 6, with different amounts of iron precursor and cashew testa extract.

| Element | Wt % | Wt % Sigma |
|---|---|---|
| C | 50.60 | 0.62 |
| O | 27.13 | 0.56 |
| Cl | 0.89 | 0.08 |
| Fe | 21.37 | 0.42 |
| Total: | 100.00 | |
| C | 36.22 | 0.36 |
| O | 39.39 | 0.32 |
| Cl | 1.14 | 0.04 |
| Fe | 23.25 | 0.23 |
| Total: | 100.00 | |
| C | 31.15 | 0.40 |
| O | 40.18 | 0.34 |
| Cl | 1.43 | 0.05 |
| Fe | 27.24 | 0.27 |
| Total: | 100.00 | |
| C | 31.25 | 0.38 |
| O | 40.38 | 0.33 |
| Cl | 0.77 | 0.04 |
| Fe | 27.61 | 0.26 |
| Total: | 100.00 | |
| C | 4.52 | 0.31 |
| O | 15.09 | 0.18 |
| Al | 0.28 | 0.05 |
| Si | 0.38 | 0.05 |
| Cl | 0.44 | 0.04 |
| Fe | 79.29 | 0.32 |
| Total: | 100.00 | |

Scanning Electron Microscope (SEM) Analysis

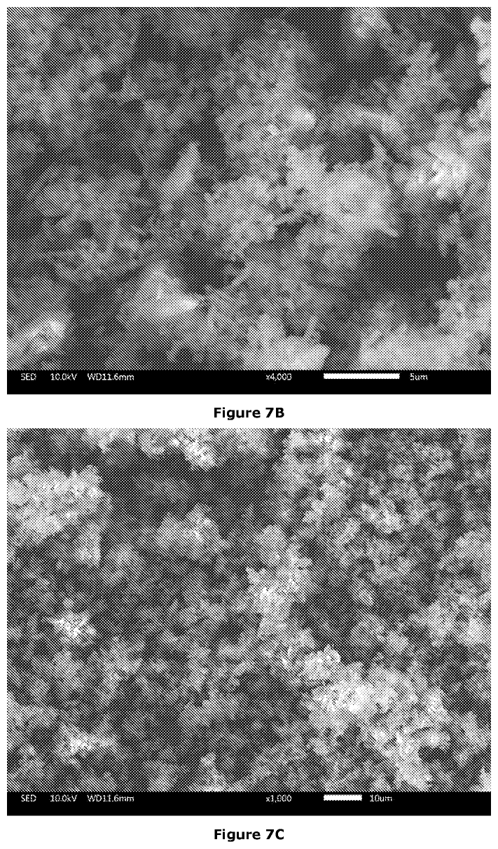

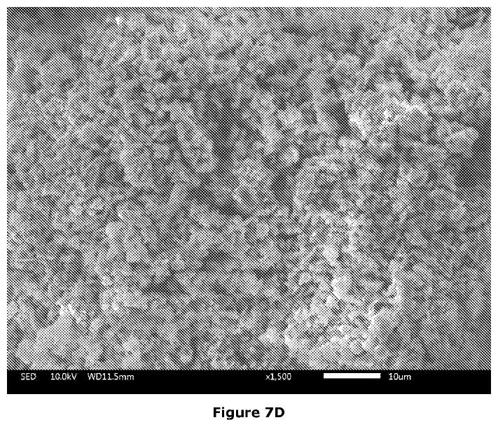

SEM results from Example 6 are shown in FIG. 7A-D.

X-ray Photoelectron Spectroscopy (XPS)

Electron Spectroscopy for Chemical Analysis (ESCA) is a surface analysis technique which analyses the elements on the sample surface, its composition, and chemical bonding state. XPS analysis of the iron-iron oxide composition showed three peaks, indicating the presence of Fe 2p, Fe²⁺ and Fe³⁺ and hence a mixed iron oxide system. Fe²⁺ can react with oxygen to form FeO which has a binding energy of about 708.4 eV. Fe³⁺ can react with oxygen to form FeOOH which has a binding energy of 710 eV or Fe₂O₃ which has a binding energy of 709.8 eV. This indicates that the reaction of cashew testa extract with iron particle and/or iron oxide particle gives an iron-iron oxide composition. It has an iron core, with an outer shell structure which contains a mixture of iron oxides. The continuous electron conductive band taking place at the shell by the iron oxides is responsible for the continuous production of reactive oxygen species (ROS) such as O₂, H₂O₂, etc is the mechanism behind the antibacterial and antiviral properties.

Formulation 1A (Hand Rub/Sanitizer)

Chitosan and cashew-iron particles were weighed into a beaker at a ratio of 15:1 to 15:14. 40 mL to 80 mL of Glycerine was added. Essential oil was added as appropriate. Acetic acid was added to make the pH of the solution between 4 to 5 along with constant stirring. The volume was made up using water.

Formulation 1B (Hand Rub/Sanitizer)

Chitosan and cashew-iron particles were weighed into a beaker at a ratio of 8:1 to 1:1. 20 mL to 80 mL of Glycerine was added. Essential oil was added as appropriate. Decyl glucoside (about 5 mL to about 20 mL) is added to improve the solubility of the essential oil in the formulation.

Formulation 2A (Disinfectant)

Chitosan and cashew-iron particles were weighed into a beaker at a ratio of 15:1 to 15:14. 0.5 g to 10 g of PEI were weighed into a beaker. Acetic acid was added to make the pH of the solution between 4 to 5 along with constant stirring. The volume was made up using water.

Formulation 2B (Disinfectant)

Chitosan and cashew-iron particles were weighed into a beaker at a ratio of 8:1 to 1:1. 0.5 g to 10 g of PEI were weighed into a beaker. Essential oil was added as appropriate. Decyl glucoside (about 5 mL to about 20 mL) is added to improve the solubility of the essential oil in the formulation. Acetic acid was added to make the pH of the solution between 4 to 5 along with constant stirring.

Formulation 3 (Disinfectant and Long Term Coating)

Chitosan and cashew-iron particles were weighed into a beaker at a ratio of 15:1 to 15:14. 0.5 g to 10 g of PEI were weighed into a beaker. 10 mL to 100 mL of Ethyl acetate was added. Acetic acid was added to make the pH of the solution between 4 to 5 along with constant stirring. The volume was made up using water.

Formulation 4A (Long Term Coating)

Chitosan and cashew-iron particles were weighed into a beaker at a ratio of 15:1 to 15:14. 0.5 g to 10 g of PEI were weighed into a beaker. 0.1% to 1% Gelatin and 0.1% to 10% 3-Glycidoxypropyltrimethoxysilane were additionally added. Acetic acid was added to make the pH of the solution between 4 to 5 along with constant stirring. The volume was made up using water.

Formulation 4B (Disinfectant and Long Term Coating)

Chitosan and cashew-iron particles were weighed into a beaker at a ratio of 8:1 to 1:1. 0.5 g to 10 g of PEI were weighed into a beaker. 20 mL to 80 mL of Glycerine was added. Essential oil was added as appropriate. Decyl glucoside (about 5 mL to about 20 mL) is added to improve the solubility of the essential oil in the formulation. 3-glycidoxypropyltrimethoxysilane (about 5 g to about 20 g) was added.

Formulation 5A (Soap for Textile)

Chitosan and cashew-iron particles were weighed into a beaker at a ratio of 30:1 to 1:300. 1 g to 20 g of cocoamphoacetate were weighed into a beaker. Acetic acid was added to make the pH of the solution between 4 to 5 along with constant stirring. The volume was made up using water.

Formulation 5B (Soap for Textile)

Chitosan and cashew-iron particles were weighed into a beaker at a ratio of 8:1 to 1:1. 20 mL to 80 mL of Glycerine was added. Essential oil was added as appropriate. Decyl glucoside (about 50 mL to about 400 mL) is added to improve the solubility of the essential oil in the formulation.

Anti-Microbial Effect of Iron Cashew Nanoparticles Against S. aureus

| | 5 min | 15 min | 1 hour | 24 hour |
|---|---|---|---|---|
| Untreated | 4.64 × 10⁷ | 4.0 × 10⁷ | 1.44 × 10⁷ | 6.8 × 10⁶ |
| | CFU/ml | CFU/ml | CFU/ml | CFU/ml |
| Iron-cashew | 6 × 10⁴ | 0 × 10⁴ | 0 × 10² | 0 |
| | CFU/ml | CFU/ml | CFU/ml | (>6 log |
| | (3 log | (>3 log | (>5 log | reduction) |
| | reduction) | reduction) | reduction) | |

Anti-Microbial Effect of Formulation Against Bacterial Cells

Formulation 1A: Hand Rub/Sanitizer

| Treated time | Log reduction in E. coli | Log reduction in S. Aureus |
|---|---|---|
| 1 min | 5 log reduction | 4 log reduction |

Formulation 2A: Disinfectant (Short-Term; ASTM E2315)

| Treated time | Log reduction in E. coli | Log reduction in S. Aureus |
|---|---|---|
| 5 minutes | 1 log reduction | 2 log reduction |
| 24 hours | >5 log reduction | >5 log reduction |

Formulation 2A: Disinfectant (Short-Term; ASTM E2180)

| Treated time | Log reduction in E. coli | Log reduction in S. Aureus |
|---|---|---|
| 30 minutes | 2 log reduction | 3 log reduction |
| 24 hours | >5 log reduction | >5 log reduction |

Quantitative Suspension Test for Evaluation of Bacterial Activity of Disinfectant

| | Initial | | | | |
|---|---|---|---|---|---|
| | suspension (N) | | | R (Log10 Reduction) = | |
| | No = (1/10N) | | | Log No − Log Na | |
| | 1.5 × 10⁸ ≤ N ≤ | Final Count Na | | Criteria: R ≥ 5.0 | |
| Test Organism | 5 × 10⁸ | 1 min | 5 min | 1 min | 5 min |
| Staphylococcus aureus | 2.0 × 10⁸ | 3.0 × 10² | 1.0 × 10² | 5.82 | 6.30 |
| ATCC 6538 | | | | | |
| Escherichia coli | 1.8 × 10⁸ | 90 | 10 | 6.31 | 7.26 |
| ATCC 10536 | | | | | |
| Pseudomonas aeruginosa | 1.5 × 10⁸ | 1.0 × 10³ | 1.0 × 10² | 5.20 | 6.20 |
| ATCC 15442 | | | | | |
| Enterococcus faecium | 3.0 × 10⁸ | 2.2 × 10³ | 6.0 × 10² | 5.14 | 5.70 |
| ATCC 6057 | | | | | |
| Salmonella typhimurium | 2.7 × 10⁸ | 5.0 × 10² | 80 | 5.73 | 6.53 |
| ATCC 13311 | | | | | |

The disinfectant satisfies the requirements of bactericidal efficacy according to EN 1276.

Formulation 3: Disinfectant (Mid-Term)

| Treated time | | Log reduction in E. coli | Log reduction in S. Aureus |
|---|---|---|---|
| 5 | minutes | 2 log reduction | 3 log reduction |
| 24 | hours | >7 log reduction | >7 log reduction |

Formulation Long Term Coating

| Treated time | | Log reduction in E. coli | Log reduction in S. Aureus |
|---|---|---|---|
| 5 | minutes | 3 log reduction | 3 log reduction |
| 24 | hours | >7 log reduction | >7 log reduction |

Formulation 4A: Long Term Coating (ASTM E2315)

| Treated time | | Log reduction in E. coli | Log reduction in S. Aureus |
|---|---|---|---|
| 5 | minutes | 4 log reduction | 4 log reduction |
| 24 | hours | >7 log reduction | >7 log reduction |

Formulation 4A: Long Term Coating (ASTM E2180)

| Treated time | Log reduction in E. coli | Log reduction in S. Aureus |
|---|---|---|
| 30 minutes | 3 log reduction | 3 log reduction |
| 24 hours | >5 log reduction | >5 log reduction |

Formulation 4A: Long Term Coating (ASTM E2180)

| Test Microorganism | Geometric mean of number of |
|---|---|
| (Bacterial Cells inoculated per test piece) | organisms in triplicate |
| Escherichia coli ATCC 8739 | 24 hrs |
| Control | 6.54 |
| Test | 2.76 |
| Percentage Reduction | >99% |
| Test Microorganism | Geometric mean of number of |
| (Bacterial Cells inoculated per test piece) | organisms in triplicate |
| Escherichia coli ATCC 8739 | 14 days |
| Control | 6.79 |
| Test | 2.18 |
| Percentage Reduction | >99% |
| Test Microorganism | Geometric mean of number of |
| (Bacterial Cells inoculated per test piece) | organisms in triplicate |
| Staphylococcus aureus ATCC 6538 | 6 Months |
| Control | 6.96 |
| Test | 2.98 |
| Percentage Reduction | >99% |
| Test Microorganism | Geometric mean of number of |
| (Bacterial Cells inoculated per test piece) | organisms in triplicate |
| Escherichia coli ATCC 8739 | 6 Months |
| Control | 6.28 |
| Test | 2.65 |
| Percentage Reduction | >99% |

Long Term Coating (JIS Z 2801)

The test were undertaken under typical laboratory conditions and consisted of inoculating a treated surface with a known concentration of S. aureus bacteria and then the reduction in bacteria was assessed. To evaluate the longevity of the antimicrobial property, the test was repeated at 30 days interval over 180 days period.

Results showed approximately 99.99% reduction in the inoculum over a 180 day duration.

To simulate the daily abrasions which would naturally occur from routine housekeeping conditions, the slides used for antimicrobial testing were cleaned and sanitized daily with multipurpose cleaning agents and wiped dry.

Notwithstanding a simulated routine cleaning and wipe down routine across the 6 month testing period, it can be concluded that an average efficacy of 99.99% reduction was achieved.

ATP Tests on Coating Durability—WOOD Floor

Result observed immediately/Day 0, after spraying on wooden floor

After spraying on wooden floor and leaving to dry totally for approx. 8 hours, ATP reading was taken (using Kikkoman LuciPac Pens and Lumitester PD30). A lower ATP reading is better as it means the solution has a higher antibacterial activity.

| | ATP count - | ATP count - | ATP count - |
|---|---|---|---|
| | immediately | Day 11 - | Day 30 - |
| | after spraying/ | with 2 | with 4 more |
| | Day 0 | times wiping | times wiping |
| Commercial long | 45 | 442 ± 62 | 375 ± 5 |
| term coating spray | | | |
| Formulation | 23 | 93 ± 4 | 213 ± 6 |
| without chitosan | | | |
| Formulation with | 9 | 73 ± 6 | 310 ± 29 |
| chitosan:cashew- | | | |
| iron particles | | | |
| ratio of 1:2 | | | |
| Formulation 1 | 10 | 119 ± 5 | 223 ± 20 |

ATP Tests on Coating Durability—Marble Floor

Result observed after spraying and wiping daily with wet cloth on marble floor for 7 days

| | ATP count | ATP count | ATP count |
|---|---|---|---|
| | on Day 7 | on Day 14 | on Day 30 |
| Commercial Big Red (BR) | 114 | 119 | 119 |
| Spray (comparator) | | | |
| Control (unwiped floor for a | 472 | 1161 | 244 |
| week)) | | | |
| Formulation without chitosan | 57 | 118 | 303 |
| Formulation with | 36 | 60 | 137 |
| chitosan:cashew-iron | | | |
| particles ratio of 1:2 | | | |
| Formulation 1 | 56 | 95 | 47 |

ATP Tests after Spraying on Plastic—Wipe Only Once Throughout for all

| | Day 11 |
|---|---|
| Control wiped with water | 1018 |
| Formulation 1 | 110 |
| BR SPRAY | 112 |

ATP Tests on Long Term Coating Durability (Formulation 4A)

| Treated | ATP reading | ATP reading after |
|---|---|---|
| Surfaces | before | 3 days and 3 wipes |
| Wood | 684 | 6 |
| Glass | 115 | 2 |
| Metal | 778 | 12 |
| Plastic | 32 | 10 |

ATP Tests on Long Term Coating Durability (One Application)

| Treated | ATP reading | ATP reading | ATP reading |
|---|---|---|---|
| Surfaces | before | after 14 days | after 30 days |
| Metal | 778 | 12 | 53 |
| Plastic | 4454 | 9 | 2 |
| Glass | 74 | 12 | 1 |
| Wood | 684 | 0 | 3 |
| Textile | 4477 | 14 | 50 |

Coomasie Brilliant Blue R Dye Degradation

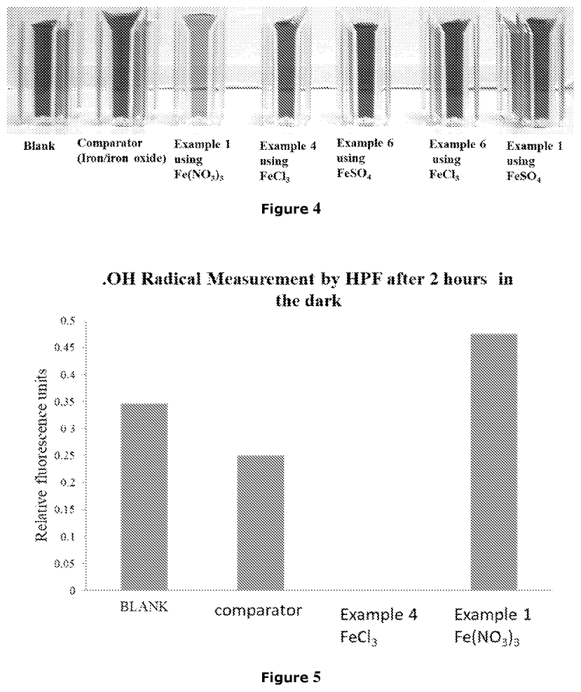

0.1 g of Iron-cashew testa nanoparticles were added to Brilliant blue dye and after 15 min incubation at room temperature the absorbance was measured at 550 nm using a UV spectrophotometer. Blank was used as the control. The results are shown in FIGS. 3 and 4.
Absorbance (arbitrary units) at 550 nm after 15 min:
Blank=3.243
Comparator (iron/iron oxide alone)=2.905
Example 1 using Fe(NO₃)₃=0.882
Example 4 using FeCl₃=2.59
Example 6 using FeSO₄=2.926
Example 6 using FeCl₃=2.87
Example 1 using FeSO₄=2.81

The results show that compared to the blank and comparator, the cashew testa extract composition (when comprising iron/iron oxide particles) is capable of degrading dyes.

Results for Test Against HCoV-229E Virus

Test Method: ISO 18184:2019 Textile Determination of Antiviral (Cashew-Iron Particles)

| | | Ig( [symbol] ) | Ig( [symbol] ) | Ig( [symbol] ) |
|---|---|---|---|---|
| Virus Types | (NO) | (IgTCID [symbol] /mL) | (IgTCID [symbol] /mL) | (IgTCID [symbol] /mL) |
| SARS-CoV-2 | 1 | 6.67 | 6.53 | 4.32 |
| MDCK | 2 | 6.63 | 6.51 | 4.35 |
| | 3 | 6.59 | 6.48 | 4.27 |

| | | | |
|---|---|---|---|
| Average Value of | 6.63 | 6.51 | 4.31 |
| IgTCID [symbol] /mL | | | |

| | |
|---|---|
| Antiviral Activity Value | 2.19 |
| Antiviral Activity Rate (%) | 99.36 |

[symbol]

indicates data missing or illegible when filed

Cashew-Iron Particles are Shown to have Anti-Viral Properties.

Test Method: EN 14476:2013+A2:2019 (Antiviral Activity of Formulations Against Human Coronavirus 229E)

The formulations were tested for their antiviral activity using Human coronavirus 229e (P1 ATCC VR-740; host MRC5 ATCC CCL-171). Pipette 1 ml of 0.3 g/L bovine albumin into a container of suitable capacity for appropriate mixing. Add 1 ml of the virus test suspension to the container, carefully avoiding the upper part of the sides. Mix. Add 8 ml of the formulation (20 g/L) to the container. Mix, start a stopwatch at once and place the container in a water bath controlled at the chosen test temperature. The activity of the product shall be determined for the contact time of 10 min. Immediately at the end of the 10 min contact time, mix, pipette 0.5 ml of the test mixture into 4.5 ml ice-cold maintenance medium and put into an ice bath. Within 30 min prepare a series of ten-fold dilutions of this mixture (test mixture+maintenance medium). Pipette tips shall be changed after each dilution to avoid carry-over of viruses. After incubation, the virus titre is calculated, reduction of virus infectivity is determined from differences of log virus titres. The infectivity is determined by the plaque assay method.

The control samples (no formulation added) show no log reduction of viral activity. The samples with formulations have at least a 4 log reduction of human coronavirus 229e in ten minutes in clean conditions.

Test Method: ISO 21702:2019 Determination of Antiviral Activity of Plastic and Other Non-Porous Surfaces (Formulation 2A)

| | | Ig( [symbol] ) | Ig( [symbol] ) | Ig( [symbol] ) |
|---|---|---|---|---|
| Virus Types | NO | (IgTCID [symbol] /mL) | (IgTCID [symbol] /mL) | (IgTCID [symbol] /mL) |
| HCoV-229E | 1 | 6.32 | 6.28 | 4.44 |
| MDCK cells | 2 | 6.37 | 6.34 | 4.46 |
| | 3 | 6.34 | 6.21 | 4.47 |
| Average Value of | | 6.34 | 6.28 | 4.46 |
| IgTCID [symbol] /mL | | | | |
| Antiviral Activity Value | | 1.88 | | |
| Antiviral Activity Rate (%) | | 98.68 | | |
| | | Ig( [symbol] ) | Ig( [symbol] ) | Ig( [symbol] ) |
| Virus Types | NO | (IgTCID [symbol] /mL) | (IgTCID [symbol] /mL) | (IgTCID [symbol] /mL) |
| HCoV-229E | 1 | 6.39 | 6.35 | 4.39 |
| MDCK cells | 2 | 6.45 | 6.42 | 4.37 |
| | 3 | 6.42 | 6.39 | 4.36 |
| Average Value of | | 6.42 | 6.39 | 4.37 |
| IgTCID [symbol] /mL | | | | |
| Antiviral Activity Value | | 2.05 | | |
| Antiviral Activity Rate (%) | | 99.10 | | |
| | | Ig( [symbol] ) | Ig( [symbol] ) | Ig( [symbol] ) |
| Virus Types | NO | (IgTCID [symbol] /mL) | (IgTCID [symbol] /mL) | (IgTCID [symbol] /mL) |
| HCoV-229E | 1 | 6.42 | 6.39 | 4.37 |
| MDCK cells | 2 | 6.46 | 6.43 | 4.36 |
| | 3 | 6.44 | 6.41 | 4.34 |

| | | | |
|---|---|---|---|
| Average Value of | 6.44 | 6.41 | 4.36 |
| IgTCID [symbol] /mL | | | |

| | |
|---|---|
| Antiviral Activity Value | 2.08 |
| Antiviral Activity Rate (%) | 99.17 |

[symbol]

indicates data missing or illegible when filed

The formulation has also been found to be effective against H3N2 virus. The formulation does not hinder the antiviral activity of the cashew-iron particles.

Disinfection of Hard, Non-Porous Surfaces Contaminated with Murine-Hepatitis Virus

Virus Treatment with dried-coated glass-slides—A coated glass-slide (treated) and non-coated glass slide (control) was prepared. 50 μl of Murine-hepatitis virus (MHV) was added onto the glass-slide and allowed it to dry completely (time took: 50 minutes) before leaving it in room-temperature for 60 mins of incubation. Glass-slides for both treated and control were then rehydrated with 2% DMEM media and supernatants were collected for viral plaque assay. Experimental protocol was made reference to ‘US EPA copper method and the prEN16777/ASTM 2197’ surface methodology.

Viral Plaque assay—H2.35 cells were used in this experiment. H2.35 cells were seeded on 24-well plates separately. MHV-treated supernatants were serially diluted 10 times to 106 and 100 μL of diluted supernatants were added to H2.35 cells. Plates were incubated for 1 h for virus binding with 15 mins rocking intervals. Plates were then washed with 1×PBS twice and 1.2% avicel were added to each well. Plates were incubated 3 days. Lastly, 1.2% avicel was removed and crystal violet was added to stain for countable plaques. Plaques were then calculated using plaque forming units per mL.

The average total virus titre of control glass-slides and treated glass-slides after 60 min is shown below.

| Non-treated | Non-treated | Non-treated | |
|---|---|---|---|
| Sample 1 | Sample 2 | Sample 3 | Average |
| 6 × 10 [symbol] | 9 × 10 [symbol] | 9 × 10 [symbol] | 8 × 10 [symbol] |
| PFU/ml | PFU/ml | PFU/ml | PFU/ml |
| Treated | Treated | Treated | |
| Sample 1 | Sample 2 | Sample 3 | Average |
| 8 × 10 [symbol] | 7 × 10 [symbol] | 4 × 10 [symbol] | 6.33 × 10 [symbol] |
| PFU/ml | PFU/ml | PFU/ml | PFU/ml |

[symbol]

indicates data missing or illegible when filed

Total log inhibition: 2.11 PFU/ml
Relative fold reduction: 126.31
Relative percentage fold reduction: 99.21%

Retention of Antiviral Properties on Treated Surface after Use

Plastic samples were treated with the formulations. Untreated plastic samples were used as control. The treated and untreated plastic samples were wiped with 300 strokes under an applied load of 1 kg as per ASTM D4828. No visible scratches/defects were observed after the wipes.

After wiping, the plastic samples were tested for their antiviral activity using Human coronavirus 229e (P1 ATCC VR-740; host MRC5 ATCC CCL-171). The untreated plastic samples show no log reduction of viral activity. The treated plastic samples have at least a 2.2 log reduction of human coronavirus 229e. The testing was conducted according to ISO 21702:2019 (Measurement of Antiviral Activity of Plastics and Other Non-porous Surfaces).

As comparison, cashew-iron particles alone were homogenously dispersed on the plastic and wiped with 300 strokes under an applied load of 1 kg as per ASTM D4828. No visible scratches/defects were observed after the wipes. When the plastic samples were tested for their antiviral activity using Human coronavirus 229e, samples show no log reduction of viral activity.

Retention of Antiviral Properties on Treated Fabric after Use

The virucidal properties of treated and untreated fabric when challenged with Human Coronavirus strain OC43 (Beta-coronavirus, ZeptoMetrix Corp. #0810024CF) was tested. Samples of treated and untreated fabric were evaluated unwashed and after 15 hand washes following 1 min, 5 min, 15 min and 60 min exposures to the virus. Test is based on ISO 18184:2019(E).

Before wash, treated fabric reduced infectivity of Coronavirus OC43 by an average of 2.4 log₁₀ (99.6%) following a 60 min exposure and may be categorized to have a good antiviral effect (≥2.0 log₁₀).

After 15 times of hand washes, treated fabric reduced infectivity of Coronavirus OC43 by an average of 3.0 log₁₀ (99.9%) following a 60 min exposure and may be categorized to have a good antiviral effect (≥2.0 log₁₀).

Untreated fabric did not show any antiviral effect.

As comparison, cashew-iron particles alone were homogenously dispersed on the fabric and evaluated unwashed and after 15 hand washes following 1 min, 5 min, 15 min and 60 min exposures to the virus. Test is based on ISO 18184:2019(E).

Before wash, cashew-iron particles alone treated fabric reduced infectivity of Coronavirus OC43 by an average of 2.4 log₁₀ (99.6%) following a 60 min exposure and may be categorized to have a good antiviral effect (≥2.0 log₁₀).

After 15 times of hand washes, cashew-iron particles alone treated fabric reduced infectivity of Coronavirus OC43 by an average of about 2 log₁₀ (99.0%) following a 60 min exposure and may be categorized to have a good antiviral effect (≥2.0 log₁₀).

Use of Formulations on Woven Fabric

Woven fabric samples were treated with the formulations. Untreated samples were used as control. The treated and untreated samples were wiped with 15,000 and 30,000 strokes as per ISO 12947 Part 2-2016 (Martindale Wear and Abrasion Tester). No visible scratches/defects were observed after the wipes.

Use of Formulations as Long Term Coating

The formulations were coated on various surfaces such as plastics, metal, painted surfaces. The formulations are shown to pass the tests for sandwich corrosion (ASTM D 1193), total immersion corrosion (ASTM F 483), low embrittling cadmium plate (ASTM F 1111), hydrogen embrittlement (ASTM F 519), flash point (ASTM D 56), effect on plastic (ASTM F 484), effect on painted surfaces (ASTM F 502), and effect on unpainted surfaces (ASTM F 485). The samples show no evidence of layering, separation, setting or crystallization when subjected to accelerated storage stability test at elevated and cold temperatures.

Antibacterial Activity of Cellulose Extracted from Durian Husk

Using ASTM E 2315 standard, 0.6 g wet durian cellulose powder were incubated with 1 mL suspension containing 10⁸ CFU/ml S. aureus for 10 minutes, then it was serial diluted and plated on Mueller Hinton agar for enumeration. 0.6 g wet weight durian cellulose showed a 0.3 log reduction of S. aureus in ten minutes.

| | | % Reduction in | Log reduction in |
|---|---|---|---|
| | CFU/mL | ten minutes | ten minutes |
| Control | 1.28 × 10⁸ | N/A | N/A |
| 0.6 g wet weight | 6.4 × 10⁷ | 50 | 0.3 |
| Durian cellulose | | | |

Spray Drying of Formulation Using Maltodextrin and Durian Cellulose as Wall Material

The formulation was mixed with 1-15% maltodextrin and 1-15% durian cellulose using a mini spray dryer (Buchi B-290, Switzerland). The inlet temperature was 120-170° C. and outlet temperature was 95-105° C. Formulations with durian cellulose conferred an additional antimicrobial activity of 0.1-1 log reduction to the resultant spray dried powder.

Levels of ·OH Generated from Iron-Cashew Particles in Water Analyzed by Hydroxyphenyl Fluorescein (HPF) Probe

Example 1 using Fe(NO₃)₃ and Example 4 using FeCl₃ were used as the samples.

0.01 g of sample was added into 1.5 mL centrifuge tubes. 1 mL of 10 μM HPF testing solution was added in each sample. The solution was well mixed by vortex and kept at room temperature in the dark. At a certain time point, the solution was centrifuged (16800 rpm×5 min), and 100 μL of solution was transferred to a black 96 well microplate for fluorescence testing. Fluorescence at 490/515 nm was collected with a microplate reader.

The type of ROS that FeNO₃-Cashew (Example 1) releases in the dark after 2 hours was ·OH radicals. FeCl₃-cashew (using Method 1) does not release ·OH radicals (FIG. 5).

Levels of ·O₂ Radical from Iron-Cashew Particles Measured by Nitroblue Tetrazolium (NBT)

Example 1 using Fe(NO₃)₃ and Example 4 using FeCl₃ were used as the samples.

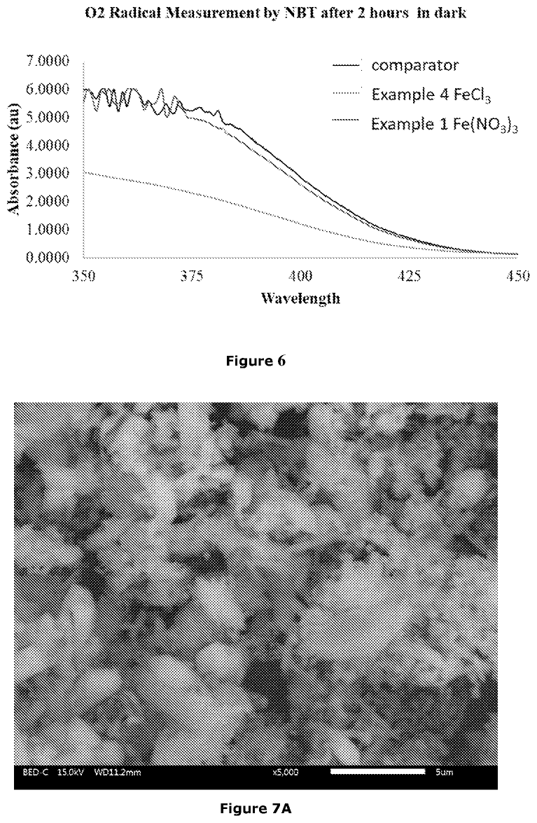

0.2 g of iron-cashew particles was added to 10 mL of 1000 mgL⁻¹ NBT in water and kept in the dark. At a certain time point, the absorption spectrum of NBT was measured by a UV-vis-NIR spectrophotometer. The absorption peak NBT continues to decrease, at 2 h, indicating the continuous generation of ·O₂ radicals by FeCl₃-cashew and react with NBT (FIG. 6).

The comparator was prepared in a similar manner. At the 2 h interval, there is no change in the absorbance value of NBT.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Throughout this specification and the claims which follow, unless the context requires otherwise, the phrase “consisting essentially of”, and variations such as “consists essentially of” will be understood to indicate that the recited element(s) is/are essential i.e. necessary elements of the invention. The phrase allows for the presence of other non-recited elements which do not materially affect the characteristics of the invention but excludes additional unspecified elements which would affect the basic and novel characteristics of the method defined.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A formulation, comprising:
a) cashew testa extract;
b) an iron particle and/or iron oxide particle; and
c) a carbohydrate;
wherein a weight ratio of the cashew testa extract to the iron particle and/or iron oxide particle is about 100:1 to about 1:200; and
wherein a weight ratio of the carbohydrate (c) to the cashew testa extract and iron particle and/or iron oxide particle (a and b) is about 8:1 to about 1:300;
wherein the cashew testa extract is at least partially incorporated in the iron particle and/or iron oxide particle.

2. The formulation according to claim 1, wherein the weight ratio of the carbohydrate (c) to the cashew testa extract and iron particle and/or iron oxide particle (a and b) is about 8:1 to about 1:1.

3. The formulation according to claim 1, wherein the weight ratio of the carbohydrate is about 1 wt % to about 15 wt % relative to the formulation.

4. The formulation according to claim 1, wherein the carbohydrate is selected from chitosan.

5. The formulation according to claim 1, wherein the weight ratio of the cashew testa extract and iron particle and/or iron oxide particle (a and b) is about 0.5 wt % to about 8 wt % relative to the formulation.

6. The formulation according to claim 1, having a pH of about 4 to about 5.

7. The formulation according to claim 1, further comprises an excipient selected from colorant, humectant, fragrance, stabilizer, permeabilizer, attachment promoter, film forming agent, transfection agent, surfactant, solvent, anti-oxidant or a combination thereof.

8. (canceled)

9. (canceled)

10. The formulation according to claim 1, further comprises a humectant selected from glycerine, urea, pyrrolidine carboxylic acid, aloe vera or a combination thereof; wherein a weight ratio of the humectant is about 2 wt % to about 60 wt % relative to the formulation.

11. (canceled)

12. The formulation according to claim 1, further comprises a fragrance, wherein the fragrance comprises an essential oil; wherein a weight ratio of the fragrance is about 0.01 wt % to about 40 wt % relative to the formulation.

13. (canceled)

14. The formulation according to claim 1, further comprises a permeabilizer selected from polyethylenimine (PEI), lactic acid, or a combination thereof; wherein a weight ratio of the permeabilizer is about 0.01 wt % to about 25 wt % relative to the formulation.

15. (canceled)

16. The formulation according to claim 1, further comprises a surfactant selected from a cocoamphoacetate salt, taurates, isethionates, olefin sulfonates, sulfosuccinates, sodium lauriminodipropionate, disodium lauroamphodiacetate, and polysorbate esters;
wherein a weight ratio of the surfactant is about 0.5 wt % to about 80 wt % relative to the formulation.

17. The formulation according to claim 1, wherein the surfactant is decyl glucoside.

18. (canceled)

19. The formulation according to claim 1, further comprising a film forming agent selected from (3-glycidyloxypropyl)trimethoxysilane and/or gelatin; wherein a weight ratio of the film forming agent is about 10 wt % to about 25 wt % relative to the formulation.

20. (canceled)

21. The formulation according to claim 1, further comprising a solvent selected from water, ethyl acetate, or a combination thereof.

22. (canceled)

23. (canceled)

24. (canceled)

25. The formulation according to claim 1, further comprising maltodextrin.

26-36. (canceled)

37. The formulation according to claim 1, for use as an antimicrobial coating, disinfectant, hand sanitizer, and/or soap; or wherein the formulation is applied on at least a surface of a fabric for use as a wet wipe.

38-41. (canceled)

42. A method of disinfecting a non-biological surface, coating a non-biological surface with an anti-microbial coating, sanitizing a biological surface or providing an anti-microbial function to a textile, comprising use of a formulation according to claim 1.

43-45. (canceled)

46. A method of forming a formulation, comprising:
a) mixing a cashew testa extract, an iron particle and/or iron oxide particle and a carbohydrate; and
b) adding an excipient selected from colorant, humectant, fragrance, stabilizer, permeabilizer, attachment promoter, film forming agent, transfection agent, surfactant, solvent, anti-oxidant or a combination thereof:
wherein a weight ratio of the cashew testa extract to the iron particle and/or iron oxide particle is about 100:1 to about 1:200; and
wherein a weight ratio of the carbohydrate to the cashew testa extract and iron particle and/or iron oxide particle is about 8:1 to about 1:300.

47. The method according to claim 46, further comprises a step after step (a) of adjusting a pH of the natural ingredient formulation to of about 4 to about 5.

48. (canceled)

49. The method according to claim 46, further comprises a step after step (a) of diluting the formulation in an aqueous medium.

50. (canceled)
